(19) United States
(12) Patent Application Publication
(10) Pub. No.: US 2023/0265517 A1
(43) Pub. Date: Aug. 24, 2023

(54) NOVEL DNA METHYLATION MARKERS ASSOCIATED WITH RENAL FUNCTION AND METHOD FOR PREDICTIING RENAL FUNCTION

(71) Applicant: The Chinese University of Hong Kong, Hong Kong (CN)

(72) Inventors: Ronald Ching-Wan MA, Hong Kong SAR (CN); Yuk Lap (Kevin) YIP, Hong Kong (CN); Yichen (Kelly) LI, Hong Kong (CN); Juliana Chung-Ngor CHAN, Hong Kong SAR (CN)

(21) Appl. No.: 18/156,945

(22) Filed: Jan. 19, 2023

Related U.S. Application Data
(60) Provisional application No. 63/300,758, filed on Jan. 19, 2022.

(51) Int. Cl. C12Q 1/6883

(52) U.S. Cl. CPC C12Q 1/6883; C12Q 2600/154; C12Q 2600/118

(57) ABSTRACT

The present application provides novel DNA methylation markers for detecting the presence or increased risk of developing diabetic kidney disease (DKD) in a subject having diabetes. The present application also provides methods and kits of diagnosing or predicting diabetic kidney disease (DKD) or a risk of suffering from DKD with these DNA methylation markers.

### CROSS-REFERENCE TO RELATED APPLICATIONS

The present application claims the priority of the U.S. provisional application No. 63/300,758, filed on Jan. 19, 2022, the entire contents of which are incorporated herein by reference.

### FIELD OF INVENTION

The present application relates to methods and kits of diagnosing or predicting a disease or condition, in particular diabetic kidney disease (DKD) and kidney failure, or a risk of suffering from DKD and kidney failure.

### BACKGROUND OF INVENTION

There is a global epidemic of type 2 diabetes, with increasing young-onset of diabetes. There is also increasing burden of kidney failure due to diabetes. This highlights the burden of diabetic kidney disease (DKD), and the need to identify individuals at risk of progression of DKD and kidney failure for early intensive interventions. Several treatments have recently been demonstrated to be helpful in retarding the progression of diabetic kidney disease, including SGLT2 inhibitors and Finerenone, which have helped to expand treatment options for diabetic kidney disease, as well as highlighting the need for tests which can help stratify those at high risk of kidney dysfunction.

There have been different efforts to identify biomarkers that can guide stratification of diabetic kidney disease, including the use of genetic and other biomarkers. Whilst genome-wide association studies (GWAS) have had considerable success in identifying genetic markers for type 2 diabetes and other complex diseases, it has had rather limited success so far in identifying loci associated with DKD. Epigenetic markers, including methylation changes and miRNA, may be able to capture the interaction between environmental factors and the genome, and may provide novel biomarkers for diabetes-related complications. Methylation markers, in particular, have been postulated to mediate the effects of metabolic memory, and hence are promising as potential biomarkers for diabetic complications. In this study, the present inventors aim to examine whether methylation at CpG sites may be associated with renal function, and whether this information can be used to predict deterioration in renal function in type 2 diabetes to identify those at risk of diabetic kidney disease.

### SUMMARY OF INVENTION

In a first aspect, provided herein is a method for determining a total methylation level of one or more CpG sites in a subject, comprising:
(a) extracting DNA from a biological sample obtained from the subject;
(b) performing an assay by contacting the DNA with reagents hybridizing to the one or more CpG sites, wherein the one or more CpG sites are selected from the group consisting of cg10272901, cg12354056, cg18461548, cg00695821, cg22822893, cg02566611, cg20741134, cg04027328, cg21573651, cg17944885, cg06449934, cg02304370, cg21919729, cg04610187 and cg18593194;
(c) detecting a total number of the one or more CpG sites based on the signals obtained from the assay; and
(d) determining the total methylation level of the one or more CpG sites using the total number.

In a second aspect, provided herein is a method for determining a total methylation level of one or more CpG sites in a subject, the method comprising:
(a) extracting DNA from a biological sample obtained from the subject;
(b) performing an assay by contacting the DNA with reagents hybridizing to the one or more CpG sites, wherein the one or more CpG sites are selected from the group consisting of those given by CpG site number provided in Table 4;
(c) detecting a total number of the one or more CpG sites based on the signals obtained from the assay; and
(d) determining the total methylation level of the one or more CpG sites using the total number.

In a third aspect, provided herein is a method for calculating a baseline eGFR or an eGFR slope in a subject, comprising:
(a) extracting DNA from a biological sample obtained from the subject;
(b) performing an assay by contacting the DNA with reagents hybridizing to two or more CpG sites, wherein the two or more CpG sites are selected from the group consisting of those given by CpG site number provided in Tables 5-6;
(c) detecting a respective number of the two or more CpG sites based on the signals obtained from the assay;
(d) determining a respective methylation level of the two or more CpG sites using the respective number; and
(e) using the respective methylation level of each CpG site multiplying respective model coefficient of the CpG site and adding up together to calculate the baseline eGFR or an eGFR slope.

In a fourth aspect, provided herein is a method for calculating a baseline eGFR or an eGFR slope in a subject, comprising:
(a) extracting DNA from a biological sample obtained from the subject;
(b) performing an assay by contacting the DNA with reagents hybridizing to two or more CpG sites, wherein the two or more CpG sites are selected from the group consisting of those given by CpG site number provided in Tables 5-6;
(c) detecting a respective number of the two or more CpG sites based on the signals obtained from the assay;
(d) determining a respective methylation level of the two or more CpG sites using the respective number; and
(e) using the respective methylation level of each CpG site multiplying respective model coefficient of the CpG site and adding up together and plus the respective intercept shown in Supplementary Tables 5-6 to calculate the baseline eGFR or an eGFR slope.

In a fifth aspect, provided herein is a kit for detecting the presence or increased risk of developing a kidney disease or kidney failure in a subject, comprising:
reagents for measuring, in a biological sample obtained from the subject, DNA methylation levels of one or more CpG sites, wherein the one or more CpG sites are selected from the group consisting of cg10272901, cg12354056, cg18461548, cg00695821, cg22822893, cg02566611, cg20741134, cg04027328, cg21573651, cg17944885, cg06449934, cg02304370, cg21919729, cg04610187 and cg18593194; and
a standard control,
wherein the presence or increased risk of developing a kidney disease or kidney failure is detected when total DNA methylation levels of the one or more CpG sites are higher or lower than the levels in the standard control.

In a sixth aspect, provided herein is a kit for detecting the presence or increased risk of developing diabetic kidney disease (DKD) in a subject having diabetes, comprising: reagents for measuring, in a biological sample obtained from the subject, DNA methylation levels of one or more CpG sites, wherein the one or more CpG sites are selected from the group consisting of those given by CpG site number provided in Table 4; and a standard control,

wherein the presence or increased risk of developing a kidney disease or kidney failure is detected when total DNA methylation levels of the one or more CpG sites are higher or lower than the levels in the standard control.

In a seventh aspect, provided herein is use of DNA methylation levels of one or more CpG sites for detecting the presence or increased risk of developing a kidney disease or kidney failure in a subject, wherein the one or more CpG sites are selected from the group consisting of cg10272901, cg12354056, cg18461548, cg00695821, cg22822893, cg02566611, cg20741134, cg04027328, cg21573651, cg17944885, cg06449934, cg02304370, cg21919729, cg04610187 and cg18593194, wherein the DNA methylation levels of one or more CpG sites are obtained from in a biological sample from the subject, and wherein the presence or increased risk of developing a kidney disease or kidney failure is detected when total DNA methylation levels of the one or more CpG sites are higher or lower than the levels in the standard control.

In an eighth aspect, provided herein is use of DNA methylation levels of one or more CpG sites for detecting the presence or increased risk of developing a kidney disease or kidney failure in a subject, wherein the CpG sites are selected from the group consisting of those given by CpG site number provided in Table 4, wherein the DNA methylation levels of one or more CpG sites are obtained from in a biological sample from the subject, and wherein the presence or increased risk of developing DKD is detected when total DNA methylation levels of the one or more CpG sites are higher or lower than the levels in the standard control.

### DESCRIPTIONS OF DRAWINGS

FIGS. 1a-1b: Distributions of eGFR and eGFR slope of the subjects. (a) Histogram of baseline eGFR in all subjects (black) and rapid decliners (defined as subjects with eGFR slope ≤−4% change of eGFR per year) (gray). (b) Distribution of eGFR slope of all subjects.

FIG. 2: Evaluation of data reproducibility. For each pair of replicated samples, the correlation of their beta values across all CpG sites was computed. The distribution of these 12 correlation values is compared with one formed by a background with 1,000 random pairs of samples.

FIG. 3: Cumulative variance explained by the top PCs of the methylation data.

FIGS. 4a-4c: Receiver-operator characteristics of the regularized logistic regression models for sex (a), age (b) and smoking status (c) constructed from the top 50 PCs of DNA methylation.

FIGS. 5a-5c: Receiver-operator characteristics of the regularized logistic regression models for eGFR constructed from the top 50 PCs of DNA methylation alone (a), sex, age and smoking status alone (b), or both (c).

FIGS. 6a-6n: Receiver-operator characteristics of the regularized logistic regression models for the other clinical variables constructed from the top 50 PCs of DNA methylation. Duration: duration of diabetes; LLD: use of lower-lipid drugs; ACEI: use of ACEI/ARB drugs; insulin: use of insulin; hypert: use of anti-hypertensive drugs. Other abbreviations are defined in the caption of Table 1.

FIGS. 7a-7d: AUROC values of the regularized logistic regression models for the four clinical variables most associated with DNA methylation at different number of PCs.

FIGS. 8a-8f: Association between CpG methylation and renal function. The methylation level of each CpG site was tested for its association with baseline eGFR (a-c) and eGFR slope (d-f). The results of all the 434,908 CpG sites analyzed in this study are shown using Manhattan plots (a,d), quantile-quantile (QQ) plots (b,e), and volcano plots (c,f). In the Manhattan plots, CpG sites with a Bonferroni-corrected p-value <0.05 are shown in grey and labeled. The horizontal grey lines show the cutoff above which all sites are significant at FDR=0.05. In the QQ plots, the diagonal straight line is the expectation under the null hypothesis. λ is the inflation factor. In the volcano plots, CpG sites with a Bonferroni-corrected p-value<0.05 are shown in dark gray.

FIGS. 9a-91: Statistical significance, in our data set, of CpG sites reported in previous studies. All panels show the same genomic locations and association p-values of the CpG sites in our study, with each panel highlighting the CpG sites reported in a particular previous study in dark gray.

FIG. 10: Correlation of methylation levels among the significantly associated CpG sites at FDR=0.05 selected by the single-site analysis. The light gray and dark gray curves show the distributions of pairwise Pearson correlation coefficients of methylation levels among the top sites for baseline eGFR and eGFR slope, respectively. The black curve shows the background distribution, formed by randomly sampling 100,000 pairs of CpG sites.

FIGS. 11a-11f: Performance of the multi-site models with different number of CpG sites. The performance of the models for baseline eGFR (a-c) and eGFR slope (d-f) was evaluated based on the Pearson correlation between the model outputs and the actual values (a,d) and the mean squared error between them (b,e), and the number of CpG sites selected as input to enter the final model was determined based on information content (c,f). In each panel, the x-axis shows the number of top CpG sites selected by the procedure for constructing the model, while the dark gray curve shows that actual number of CpG sites with a non-zero coefficient. The vertical dotted lines show the final models determined according to the information content.

FIGS. 12a-12f: Performance of the multi-site models constructed from and applied to the primary cohort. Scatter plots of predicted baseline eGFR (a,b) and eGFR slope (d,e) against their corresponding actual measurements using selected CpG sites with (a,d) or without (b,e) the covariates. In Panels a-b and d-e, the black dashed lines mark the diagonal on which the predicted and actual values would be the same. Comparison of the baseline eGFR (c) and eGFR slope (f) multi-site models with alternative models that involve either only CpG sites with Bonferroni-corrected single-site p-values <0.05, only CpG sites statistically significant at FDR=0.05 in the single-site analysis, or only the set of CpG sites with most significant single-site p-values, with the set size equals the number of sites selected in the final multi-site model. In Panels c and f, the results are based on 5-fold cross-validation and the horizontal dash lines show the Pearson correlations of models with only covariates as input.

FIGS. 13a-13d: Performance of the multi-site models with the same number of CpG sites as in the real models but randomly selected. The blue bars show the histograms of Pearson correlation coefficients between the actual and predicted baseline eGFR (a-b) and eGFR slope (c-d) of these random models with (a,c) or without (b,d) allowing covariates in the models. The read dashed curves show the fitted normal distributions. The vertical dash lines show the Pearson correlations of the actual models constructed by our procedure. Some random eGFR slope models without allowing covariates had none of the CpG sites with a non-zero coefficient, and thus these models always predicted the same eGFR slope values, leading to a Pearson correlation of 0 with the actual eGFR slopes.

FIGS. 14a-14d: Performance of the multi-site models constructed from the primary cohort and applied to an independent Pima Indian cohort. Scatter plots of predicted baseline eGFR (a-b) or eGFR slope (c-f) against their corresponding actual measurements using selected CpG sites with (a,c,e) or without (b,d,f) the covariates. In all panels, the black dashed lines mark the diagonal on which the predicted and actual values would be the same.

FIG. 15: Support for the functional significance of genes near the CpG sites identified in our single-site and multi-site analyses. Each row corresponds to a CpG site and all genes within 1 kb from it. The “Single-site” and “Multi-site” columns show whether a site is significant at FDR=0.05 in our single-site analysis and whether it is included in the final multi-site model, respectively. The “DNAm” and “DEGs” columns show whether at least one of the nearby genes is differentially methylated or differentially expressed in samples with and without kidney function decline in one or more previous methylation or gene expression studies, respectively. The “eQTL” column shows whether at least one of the nearby genes is associated with an expression quantitative trait locus identified in human kidney samples in a previous study. The “MarkerGenes” column shows whether at least one of the nearby genes is a cell type-specific marker of a major kidney cell type as identified previously. Only CpG sites where the nearby genes have at least 3 and 1 functional supports, respectively for baseline eGFR and eGFR slope, are shown.

FIG. 16: Training, parameter tuning and evaluation procedures of the multi-site model. All samples are split into an overall training set (90%) and an overall testing set (10%). The training set is used to assign weights to each CpG site using a 10-fold cross-validation procedure repeated for 10 times. Models are then trained using all samples in the overall training set as examples and different numbers of highest-weight CpG sites as features. The best model is selected using a BIC criterion. It is then applied to the samples in the overall testing set to evaluate model performance. A final model is also constructed using the same procedure but with all 100% samples assigned to the overall training set. This model is evaluated using data from the Pima Indian cohort.

FIGS. 17a-17f: Functional significance of our selected CpG sites' methylation levels in kidney. Methylation levels of cg21573651 (a-c) and cg04610187 (d-e) in kidney samples are significantly different between kidney disease (CKD/DKD) patients and control groups (a, d). They also correlate significantly with eGFR (b, e) and fibrosis (c, f). P-values were computed using two-sided test based on asymptotic t approximation. Con: healthy control. HTN: hypertension.

### DETAILED DESCRIPTIONS

In this disclosure, the term “type 2 diabetes” (T2D) refers to a metabolic disorder that is characterized by high blood glucose in the context of varying combinations of insulin resistance and insulin deficiency. Type 2 diabetes may be caused by a combination of lifestyle and genetic factors. Diabetes can be caused by distinct clinical entities such as endocrine disorders (e.g., Cushing's syndrome) and chronic pancreatitis. However, the majority of people with diabetes have risk factors including but not limited to obesity, hypertension, high blood cholesterol, metabolic syndrome (high triglyceride, low HDL-C, high blood glucose, high blood pressure, large waist), which may share common metabolic pathways, further amplified by aging, energy dense diets (e.g., high-fat and high glucose), sedentary lifestyle and use of certain drugs (e.g., beta blockers, steroids). On the other hand, having relatives (especially first degree) with T2D increases risks of developing T2D substantially. Symptoms of T2D often include polyuria (frequent urination), polydipsia (increased thirst), polyphagia (increased hunger), fatigue, and weight loss. The abnormal neurohormonal and metabolic milieu characterized by hyperglycemia, dyslipidemia and low-grade inflammation can trigger a cascade of signaling pathways, which can lead to cell death and dysregulated cell growth, giving rise to multiple morbidities including heart disease, strokes, limb amputation, visual loss, kidney failure, cancers, and cognitive impairment.

In this disclosure, the term “diabetic kidney disease (DKD)” is proteinuria, usually also associated with a progressive decrease in glomerular filtration rate (GFR) caused by long-term diabetes. Diabetic kidney disease is one of the most important complications of diabetic patients. The incidence rate worldwide is also on the rise, and it has become the second cause of end-stage renal disease. Due to its complex metabolic disorders, once it develops into end-stage renal disease, it is often more difficult than the treatment of other kidney diseases, so timely prevention and treatment is of great significance to delaying diabetic kidney disease.

In this disclosure, the term “biological sample” or “sample” includes any section of tissue or bodily fluid taken from a test subject such as a biopsy and autopsy sample, and frozen section taken for histologic purposes, or processed forms of any of such samples. Biological samples include blood and blood fractions or products (e.g., serum, plasma, platelets, white blood cells, red blood cells, and the like), sputum or saliva, lymph and tongue tissue, cultured cells, e.g., primary cultures, explants, and transformed cells, stool, urine, stomach biopsy tissue etc., A biological sample is typically obtained from an eukaryotic organism, which may be a mammal, may be a primate and may be a human subject.

The term “DNA methylation level” refers to the extent to which a CpG site is methylated in a sample obtained from an individual. A CpG site at a locus can be fully or partially methylated, and the pattern of methylation can be random, uniform, or specific to portions of the CpG site. Moreover, the pattern and extent of methylation of a CpG site can vary, for example between chromosomes in the same cell, tissues of the same individual, or different individuals. Thus, measuring a DNA methylation level in a sample can provide a detailed methylation pattern and can reflect the context in which the sample was obtained. The measured DNA methylation level can be used to determine whether a CpG site is differentially methylated, for example between T2D-positive and T2D-negative individuals. In the case of individual CpG sites, in each cell there are only up to two copies (due to the diploid genome) and thus there are only three possibilities: both methylated, exactly one methylated, or both unmethylated. The methylation level of the CpG site actually refers to the proportion of measured copies from different cells that are methylated.

In this disclosure, the term “standard control” refers to a sample suitable for the use of a method of the present invention, in order to quantitatively determine the level of expression (e.g., abundance of RNA transcripts or gene products) or DNA methylation in a test sample for one or more genomic regions of interest (for example, a gene or genomic locus). The standard control contains a known level or levels of expression or DNA methylation for the genomic region(s) of interest, such that the levels closely reflect those of an average healthy individual not suffering from T2D and not at an increased risk of later developing T2D. The standard control may be derived from one or more healthy individuals.

“Higher or lower than levels in a standard control” as used herein refers to differences between the level of expression or DNA methylation in test sample as compared with corresponding levels in a standard control, for the same CpG sites of interest. Our single-site and multi-site models in the invention both take numeric methylation levels (between 0 and 1) as input. A higher level is higher numeric methylation levels of one or more CpG sites compared to the levels of the corresponding one or more CpG sites in the standard control. Similarly, a lower level is lower numeric methylation levels of one or more CpG sites compared to the levels of the corresponding one or more CpG sites in the standard control.

The term “subject” or “subject in need of treatment,” as used herein includes individuals who seek medical attention due to risk of, or actual suffering from diabetes such as T2D or diabetes-related complications such as DKD. Subjects also include individuals currently undergoing therapy that seek manipulation of the therapeutic regimen. Subjects or individuals in need of treatment include those that demonstrate symptoms of diabetes such as T2D or diabetes-related complications such as DKD, or are at risk of suffering from diabetes such as T2D or diabetes-related complications such as DKD or related symptoms. For example, a subject in need of treatment includes individuals with a genetic predisposition or family history for diabetes or diabetes-related complications, those who have suffered relevant symptoms in the past, those who have been exposed to a triggering substance or event, as well as those suffering from chronic or acute symptoms of the condition. A “subject in need of treatment” may be at any age of life.

The term “cutoff” as used herein can refer to a predetermined value. Taking baseline eGFR for an example, if the measured baseline eGFR of a subject is below the predetermined cutoff, such as eGFR<60 ml/min/1.73 m2, it indicates that the subject has increased risk of having a kidney disease, such as DKD. As for baseline eGFR and eGFR slope, the cutoff can be conventionally determined by a person skilled in the art.

In a first aspect, provided herein is a method for determining a total methylation level of one or more CpG sites in a subject, comprising:
(a) extracting DNA from a biological sample obtained from the subject;
(b) performing an assay by contacting the DNA with reagents hybridizing to the one or more CpG sites, wherein the one or more CpG sites are selected from the group consisting of cg10272901, cg12354056, cg18461548, cg00695821, cg22822893, cg02566611, cg20741134, cg04027328, cg21573651, cg17944885, cg06449934, cg02304370, cg21919729, cg04610187 and cg18593194;
(c) detecting a total number of the one or more CpG sites based on the signals obtained from the assay; and
(d) determining the total methylation level of the one or more CpG sites using the total number.

In some embodiments, the subject has already had diabetes, such as type 1 diabetes (T1D) or type 2 diabetes (T2D).

In some embodiments, the reagents hybridizing to the one or more CpG sites are those involved in methods selected from the group consisting of High-performance Liquid Chromatography (HPLC), High-performance Capillary Electrophoresis (HPCE), methylation-sensitive restriction Endonuclease-PCR/Southern (MSRE-PCR/Southern), MethyLight, Pyrosequencing, combined bisulfite restriction analysis (COBRA), methylation-specific PCR (MSP), bisulfite sequencing, high resolution melting (HRM), Restriction Landmark Genomic Scanning (RLGS), amplification of inter-methylated sites (AIMS), Methylated CpG-island amplification (MCA), Differential Methylation Hybridization (DMH), HpaII tiny fragment Enrichment by Ligation-mediated PCR (HELP), Methylated DNA immunoprecipitation (MeDIP) and other technologies for evaluating methylation level.

In some embodiments, the biological sample may be selected from the group consisting of blood, serum, plasma, sputum, saliva, kidney biopsy tissue, urine and the like.

In some embodiments, the subject is of Asian descent, preferably a Chinese.

In some embodiments, if the total DNA methylation level is higher or lower than the corresponding total level in a standard control, the method further comprising administering to the subject agents for reducing blood glucose and urine protein. The standard control may be a corresponding biological sample obtained from a healthy subject having no diabetes. The agents for reducing blood glucose and urine protein may include, but not limited to metformin hydrochloride, acarbose, empagliflozin, dapagliflozin, canagliflozin, ertugliflozin, GLP-1 agonists such as liraglutide, exenatide, dulaglutide, semaglutide and similar drugs, ACEI classes such as benazepril hydrochloride, and ARB classes such as losartan potassium, telmisartan, irbesartan, and the like, or mineralocorticoid receptor antagonists such as finenrenone and the like.

In a second aspect, provided herein is a method for determining a total methylation level of one or more CpG sites in a subject, the method comprising:
(a) extracting DNA from a biological sample obtained from the subject;
(b) performing an assay by contacting the DNA with reagents hybridizing to the one or more CpG sites, wherein the one or more CpG sites are selected from the group consisting of those given by CpG site number provided in Table 4;
(c) detecting a total number of the one or more CpG sites based on the signals obtained from the assay;
(d) determining the total methylation level of the one or more CpG sites using the total number.

In some embodiments, the one or more CpG sites are selected from the group consisting of those having a positive value of the Model coefficient in Table 4, and if the total DNA methylation level is lower than the corresponding total level in a standard control, the method further comprising administering to the subject agents for reducing blood glucose and urine protein.

In some embodiments, the one or more CpG sites are selected from the group consisting of those having a negative value of the Model coefficient in Table 4, and if the total DNA methylation level is higher than the corresponding total level in a standard control, the method further comprising administering to the subject agents for reducing blood glucose and urine protein.

In some embodiments, the subject has already had diabetes, such as type 1 diabetes (T1D) or type 2 diabetes (T2D).

In some embodiments, the reagents hybridizing to the one or more CpG sites are those involved in methods selected from the group consisting of High-performance Liquid Chromatography (HPLC), High-performance Capillary Electrophoresis (HPCE), methylation-sensitive restriction Endonuclease-PCR/Southern (MSRE-PCR/Southern), MethyLight, Pyrosequencing, combined bisulfite restriction analysis (COBRA), methylation-specific PCR (MSP), bisulfite sequencing, high resolution melting (HRM), Restriction Landmark Genomic Scanning (RLGS), amplification of inter-methylated sites (AIMS), Methylated CpG-island amplification (MCA), Differential Methylation Hybridization (DMH), HpaII tiny fragment Enrichment by Ligation-mediated PCR (HELP), Methylated DNA immunoprecipitation (MeDIP) and other technologies for evaluating methylation level.

In some embodiments, the subject is of Asian descent, preferably a Chinese.

In an embodiment, the standard control may be a corresponding biological sample obtained from a healthy subject having no diabetes. The agents for reducing blood glucose and urine protein may include, but not limited to metformin hydrochloride, acarbose, empagliflozin, dapagliflozin, canagliflozin, ertugliflozin, GLP-1 agonists such as liraglutide, exenatide, dulaglutide, semaglutide and similar drugs, ACEI classes such as benazepril hydrochloride, and ARB classes such as losartan potassium, telmisartan, irbesartan, and the like, or mineralocorticoid receptor antagonists such as finenrenone and the like.

In some embodiments, the biological sample may be selected from the group consisting of blood, serum, plasma, sputum, saliva, kidney biopsy tissue and urine.

In a third aspect, provided herein is a method for calculating a baseline eGFR or an eGFR slope, comprising:
(a) extracting DNA from a biological sample obtained from the subject;
(b) performing an assay by contacting the DNA with reagents hybridizing to two or more CpG sites, wherein the two or more CpG sites are selected from the group consisting of those given by CpG site number provided in Tables 5-6;
(c) detecting a respective number of the two or more CpG sites based on the signals obtained from the assay;
(d) determining a respective methylation level of the two or more CpG sites using the respective number; and
(e) using the respective methylation level of each CpG site multiplying respective model coefficient of the CpG site and adding up together to calculate the baseline eGFR or an eGFR slope.

In some embodiments, for the baseline eGFR, the two or more CpG sites are selected from the group consisting of those given by CpG site number provided in Tables 5 and the respective model coefficient is selected from the group consisting of that shown in “with covariates” and that shown in “without covariates” corresponding to each CpG sites shown in Table 5, and/or for the eGFR slope, two or more CpG sites are selected from the group consisting of those given by CpG site number provided in Table 6 and the respective model coefficient is selected from the group consisting of that shown in “with covariates” and that shown in “without covariates” corresponding to each CpG sites shown in Table 6. For the supplementary Table 5, left table shows baseline eGFR without covariate and right table shows baseline eGFR with covariate, and for the supplementary Table 6, left table shows eGFR slope without covariate and right table shows eGFR slope with covariate.

In some embodiments, the method further comprises comparing the baseline eGFR or the eGFR slope to a cutoff, and wherein if the baseline eGFR or the eGFR slope is below the cutoff, the method further comprising administering to the subject agents for reducing blood glucose and urine protein.

The agents for reducing blood glucose and urine protein may include, but not limited to metformin hydrochloride, acarbose, empagliflozin, dapagliflozin, canagliflozin, ertugliflozin, GLP-1 agonists such as liraglutide, exenatide, dulaglutide, semaglutide and similar drugs, ACEI classes such as benazepril hydrochloride, and ARB classes such as losartan potassium, telmisartan, irbesartan, and the like, or mineralocorticoid receptor antagonists such as finenrenone and the like.

In some embodiments, the subject has already had diabetes, such as type 1 diabetes (T1D) or type 2 diabetes (T2D).

In some embodiments, the reagents hybridizing to the one or more CpG sites are those involved in methods selected from the group consisting of High-performance Liquid Chromatography (HPLC), High-performance Capillary Electrophoresis (HPCE), methylation-sensitive restriction Endonuclease-PCR/Southern (MSRE-PCR/Southern), MethyLight, Pyrosequencing, combined bisulfite restriction analysis (COBRA), methylation-specific PCR (MSP), bisulfite sequencing, high resolution melting (HRM), Restriction Landmark Genomic Scanning (RLGS), amplification of inter-methylated sites (AIMS), Methylated CpG-island amplification (MCA), Differential Methylation Hybridization (DMH), HpaII tiny fragment Enrichment by Ligation-mediated PCR (HELP), Methylated DNA immunoprecipitation (MeDIP) and other technologies for evaluating methylation level.

In some embodiments, the biological sample may be selected from the group consisting of blood, serum, plasma, sputum, kidney biopsy tissue, saliva, urine and the like.

In some embodiments, the subject is of Asian descent.

In some embodiments, the subject is a Chinese.

In a fourth aspect, provided herein is a method for calculating a baseline eGFR or an eGFR slope in a subject, comprising:
(a) extracting DNA from a biological sample obtained from the subject;
(b) performing an assay by contacting the DNA with reagents hybridizing to two or more CpG sites, wherein the two or more CpG sites are selected from the group consisting of those given by CpG site number provided in Tables 5-6;
(c) detecting a respective number of the two or more CpG sites based on the signals obtained from the assay;
(d) determining a respective methylation level of the two or more CpG sites using the respective number; and
(e) using the respective methylation level of each CpG site multiplying respective model coefficient of the CpG site and adding up together and plus the respective intercept shown in Supplementary Tables 5-6 to calculate the baseline eGFR or an eGFR slope.

In some embodiments, for the baseline eGFR, the two or more CpG sites are selected from the group consisting of those given by CpG site number provided in Tables 5 and the respective model coefficient is selected from the group consisting of that shown in “with covariates” and that shown in “without covariates” corresponding to each CpG sites shown in Table 5, and/or for the eGFR slope, two or more CpG sites are selected from the group consisting of those given by CpG site number provided in Table 6 and the respective model coefficient is selected from the group consisting of that shown in “with covariates” and that shown in “without covariates” corresponding to each CpG sites shown in Table 6. For the supplementary Table 5, left table shows baseline eGFR without covariate and right table shows baseline eGFR with covariate, and for the supplementary Table 6, left table shows eGFR slope without covariate and right table shows eGFR slope with covariate.

In some embodiments, if covariates are considered, during the calculation of the baseline eGFR or the eGFR slope, the step (e) is using the methylation level of each CpG site multiplying respective model coefficient of the CpG site and using the covariate multiplying respective coefficient such as those shown in Supplementary Tables 5 and 6, and adding up together and plus the respective intercept shown in Supplementary Tables 5-6 to calculate a baseline eGFR or an eGFR slope.

In some embodiments, the method further comprises comparing the baseline eGFR or the eGFR slope to a cutoff, and wherein if the baseline eGFR or the eGFR slope is below the cutoff, the method further comprising administering to the subject agents for reducing blood glucose and urine protein.

The agents for reducing blood glucose and urine protein may include, but not limited to metformin hydrochloride, acarbose, empagliflozin, dapagliflozin, canagliflozin, ertugliflozin, GLP-1 agonists such as liraglutide, exenatide, dulaglutide, semaglutide and similar drugs, ACEI classes such as benazepril hydrochloride, and ARB classes such as losartan potassium, telmisartan, irbesartan, and the like, or mineralocorticoid receptor antagonists such as finenrenone and the like.

In some embodiments, the subject has already had diabetes, such as type 1 diabetes (T1D) or type 2 diabetes (T2D).

In some embodiments, the reagents hybridizing to the one or more CpG sites are those involved in methods selected from the group consisting of High-performance Liquid Chromatography (HPLC), High-performance Capillary Electrophoresis (HPCE), methylation-sensitive restriction Endonuclease-PCR/Southern (MSRE-PCR/Southern), MethyLight, Pyrosequencing, combined bisulfite restriction analysis (COBRA), methylation-specific PCR (MSP), bisulfite sequencing, high resolution melting (HRM), Restriction Landmark Genomic Scanning (RLGS), amplification of inter-methylated sites (AIMS), Methylated CpG-island amplification (MCA), Differential Methylation Hybridization (DMH), HpaII tiny fragment Enrichment by Ligation-mediated PCR (HELP), Methylated DNA immunoprecipitation (MeDIP) and other technologies for evaluating methylation level.

In some embodiments, the biological sample may be selected from the group consisting of blood, serum, plasma, sputum, saliva, kidney biopsy tissue, urine and the like.

In some embodiments, the subject is of Asian descent.

In some embodiments, the subject is a Chinese.

In some embodiments, the method further comprises determining the risk factors of the subject selected from the group consisting of sex, age, smoking status, duration of diabetes and family history of diabetes.

In a fifth aspect, provided herein is a kit for detecting the presence or increased risk of developing kidney disease or kidney failure in a subject, comprising:
reagents for measuring, in a biological sample obtained from the subject, DNA methylation levels of one or more CpG sites, wherein the one or more CpG sites are selected from the group consisting of cg10272901, cg12354056, cg18461548, cg00695821, cg22822893, cg02566611, cg20741134, cg04027328, cg21573651, cg17944885, cg06449934, cg02304370, cg21919729, cg04610187 and cg18593194; and
a standard control,
wherein the presence or increased risk of developing a kidney disease or kidney failure is detected when total DNA methylation levels of the one or more CpG sites are higher or lower than the levels in the standard control.

In a sixth aspect, provided herein is a kit for detecting the presence or increased risk of developing kidney disease or kidney failure in a subject, comprising: reagents for measuring, in a biological sample obtained from the subject, DNA methylation levels of one or more CpG sites, wherein the one or more CpG sites are selected from the group consisting of those given by CpG site number provided in Table 4; and
a standard control,
wherein the presence or increased risk of developing a kidney disease or kidney failure is detected when total DNA methylation levels of the one or more CpG sites are higher or lower than the levels in the standard control.

In some embodiments, the reagents are used for measuring DNA methylation levels of one or more CpG sites selected from the group consisting of those having a positive value of the Model coefficient in Table 4, and wherein the subject has a kidney disease or kidney failure or increased risk of developing a kidney disease or kidney failure if the DNA methylation levels are lower than the levels in the standard control.

In some embodiments, the reagents are used for measuring the DNA methylation levels of the CpG sites selected from the group consisting of those having a negative value of the Model coefficient in Table 4, and wherein the subject has a kidney disease or kidney failure or increased risk of developing a kidney disease or kidney failure if the DNA methylation levels are higher than the levels in the standard control.

In some embodiments, the subject has already had diabetes, such as type 1 diabetes (T1D) or type 2 diabetes (T2D). Optionally, the kidney disease mentioned above may be diabetic kidney disease (DKD).

In some embodiments, the kit further comprises reagents for measuring the DNA methylation levels, the reagents comprise those for performing the methods selected from the group consisting of High-performance Liquid Chromatography (HPLC), High-performance Capillary Electrophoresis (HPCE), methylation-sensitive restriction Endonuclease-PCR/Southern (MSRE-PCR/Southern), MethyLight, Pyrosequencing, combined bisulfite restriction analysis (COBRA), methylation-specific PCR (MSP), bisulfite sequencing, high resolution melting (HRM), Restriction Landmark Genomic Scanning (RLGS), amplification of inter-methylated sites (AIMS), Methylated CpG-island amplification (MCA), Differential Methylation Hybridization (DMH), HpaII tiny fragment Enrichment by Ligation-mediated PCR (HELP), Methylated DNA immunoprecipitation (MeDIP) and other technologies for evaluating methylation level.

In some embodiments, the biological sample may be selected from the group consisting of blood, serum, plasma, sputum, saliva, kidney biopsy tissue, urine and the like.

In some embodiments, the subject is of Asian descent.

In some embodiments, the subject is a Chinese.

In a seventh aspect, provided herein is use of DNA methylation levels of one or more CpG sites for detecting the presence or increased risk of developing a kidney disease or kidney failure in a subject, wherein the one or more CpG site are selected from the group consisting of cg10272901, cg12354056, cg18461548, cg00695821, cg22822893, cg02566611, cg20741134, cg04027328, cg21573651, cg17944885, cg06449934, cg02304370, cg21919729, cg04610187 and cg18593194, wherein the DNA methylation levels of one or more CpG sites are obtained from in a biological sample from the subject, and wherein the presence or increased risk of developing a kidney disease or kidney failure is detected when total DNA methylation levels of the one or more CpG sites are higher or lower than the levels in the standard control.

In an eighth aspect, provided herein is use of DNA methylation levels of one or more CpG sites for detecting the presence or increased risk of developing a kidney disease or kidney failure in a subject, wherein the one or more CpG sites are selected from the group consisting of those given by CpG site number provided in Table 4, wherein the DNA methylation levels of one or more CpG sites are obtained from in a biological sample from the subject, and wherein the presence or increased risk of developing a kidney disease or kidney failure is detected when total DNA methylation levels of the one or more CpG sites are higher or lower than the levels in the standard control.

In some embodiments, the one or more CpG sites are selected from the group consisting of those having a positive value of the Model coefficient in Table 4, and wherein the subject has a kidney disease or kidney failure or increased risk of developing a kidney disease or kidney failure if the DNA methylation levels are lower than the levels in the standard control.

In some embodiments, the one or more CpG sites are selected from the group consisting of those having a negative value of the Model coefficient in Table 4, and wherein the subject has a kidney disease or kidney failure or increased risk of developing a kidney disease or kidney failure if the DNA methylation levels are higher than the levels in the standard control.

In some embodiments, the subject has already had diabetes, such as type 1 diabetes (T1D) or type 2 diabetes (T2D). Optionally, the kidney disease mentioned above may be diabetic kidney disease (DKD).

In some embodiments, the DNA methylation levels are measured by methods selected from the group consisting of High-performance Liquid Chromatography (HPLC), High-performance Capillary Electrophoresis (HPCE), methylation-sensitive restriction Endonuclease-PCR/Southern (MSRE-PCR/Southern), MethyLight, Pyrosequencing, combined bisulfite restriction analysis (COBRA), methylation-specific PCR (MSP), bisulfite sequencing, high resolution melting (HRM), Restriction Landmark Genomic Scanning (RLGS), amplification of inter-methylated sites (AIMS), Methylated CpG-island amplification (MCA), Differential Methylation Hybridization (DMH), HpaII tiny fragment Enrichment by Ligation-mediated PCR (HELP) and Methylated DNA immunoprecipitation (MeDIP) and other technologies for evaluating methylation level.

In some embodiments, the biological sample may be selected from the group consisting of blood, serum, plasma, sputum, saliva, kidney biopsy tissue, urine and the like.

In some embodiments, the subject is of Asian descent.

In some embodiments, the subject is a Chinese.

### EXAMPLES

The following examples are provided by way of illustration only and not by way of limitation. Those of skill in the art will readily recognize a variety of non-critical parameters that could be changed or modified to yield essentially the same or similar results.

Materials and Methods

Participants Recruitment and Clinical Variable Measurements

We included subjects from the Hong Kong Diabetes Register (HKDR), which was established at the Prince of Wales Hospital, the teaching hospital of the Chinese University of Hong Kong. The HKDR consecutively enrolled patients who were referred to the Diabetes Mellitus and Endocrine Centre for comprehensive assessment of complications and metabolic control, including patients referred from specialty clinics, community clinics and general practitioners. All enrolled subjects underwent extensive clinical evaluation at baseline as well as follow-up for development of diabetes complications. Ethical approval was obtained from the Clinical Research Ethics Committees of the Chinese University of Hong Kong. Written informed consent was obtained from all subjects at the time of enrolment for collection of clinical information and biosamples for archival and research purposes.

Details of the cohort and assessment have been described in detail in previous publications. In brief, subjects with diabetes were evaluated as part of a structured assessment for diabetes complications according to a modified European DiabCare protocol. All patients in the HKDR underwent clinical assessments and laboratory investigations after 8-hour overnight fast, including eye, feet, urine and blood examinations. Eye examination included visual acuity and fundoscopy through dilated pupils or retinal photography. Retinopathy was defined by typical changes due to diabetes, laser scars, or a history of vitrectomy. Foot examination was performed using Doppler ultrasound scan and monofilament and graduated tuning fork. Fasting blood was sampled for measurement of plasma glucose, HbA1c, lipid profile (total cholesterol, high-density lipoprotein [HDL] cholesterol, triglycerides and calculated low-density lipoprotein [LDL] cholesterol), and random spot urinary sample was used to assess albumin to creatinine ratio (ACR). The Chronic Kidney Disease Epidemiology Collaboration (CKD-EPI) equation was used to estimate glomerular filtration rate.

Clinical outcomes were defined using hospital discharge diagnoses based on the International Classification of Diseases, Ninth Revision (ICD-9) and mortality as censored on or before Jun. 30, 2014. The Hong Kong Hospital Authority Central Computer System records admissions to all public hospitals, which provides about 95% of inpatient bed-days in Hong Kong. All hospitalization records were retrieved from this system using a unique identifier number. Results of follow-up investigations including eGFR were likewise retrieved for each subject from the electronic health record from the Central Computer System.

Between 1995 and Dec. 31, 2007, a consecutive cohort consisting of 10,129 patients with diabetes was assessed, with follow-up. For the current analysis, we created a nested case control cohort based on incident diabetic kidney disease (defined according to the censor date of Jun. 30, 2014, around the time when the EWAS was initiated when the case-control status was defined), matched according to age at baseline. All subjects were selected based on being free of known cardiovascular events at baseline. In addition to use of the clinical data with regard to baseline renal function, we retrieved follow-up laboratory data up to Jun. 30, 2017, in order to calculate the eGFR slope during follow-up for each individual, up to the censor date, eGFR<15 ml/min/1.73 m² or death, whichever event occurs sooner.

eGFR slope was determined by fitting the following linear mixed model:

log(eGFRᵢⱼ)=βₒ+β₁tᵢⱼ+boi+b₁ᵢtᵢⱼ+Eᵢⱼ, (1)

where log(eGFRᵢⱼ) is the log-transformed eGFR of i-th individual at j-th measurement, tᵢⱼ is the time for measuring eGFRᵢⱼ, β₀ and β₁ are coefficients for the fixed effects while b₀ᵢ and b₁ᵢ are coefficients for the random effects that are specific to the i-th individual, and Eᵢⱼ is the random noise.

After fitting the model, the individual-specific slope is given by the following:

(eGFR slope)ᵢ=(e^{β1+}^{^{b}}¹ⁱ−1)×100, (2)

which is expressed as the percentage change of eGFR per year.

DNA Methylation Data Production and Processing

Whole blood was taken at the baseline assessment visit in a fasting state. Genomic DNA from leukocytes was extracted using traditional phenol-chloroform methods and quantified using Picogreen. Bisulfite conversion was performed using EZGold Methylation kit (Zymo), as per standard protocol. After DNA extraction and bisulfite treatment, DNA methylation in each sample was measured using the Illumina Infinium HumanMethylation450K Beadchip, which covered around 485,000 CpG sites across the genome.

The RnBeads package (version 1.6.1) was used to preprocess the raw data. First, 10,119 sites were removed because they overlapped with single nucleotide polymorphisms (SNPs). Probes and samples with a large fraction of unreliable measurements, defined as those with detection p-values larger than 0.05, were also removed. Furthermore, probes in contexts other than CpG sites and probes on sex chromosomes were removed. Background correction was then conducted using the “noob” method in the methylumi package (version 2.20.0) and the signal intensities were normalized using the SWAN method in the minfi package (version 1.20.2). After these filtering and normalization steps, 453,128 probes and 1,268 samples remained. In all downstream analyses, we also excluded probes with missing methylation values in any sample, resulting in the final number of 434,908 probes. In the whole study, genomic coordinates were based on the reference human genome hg19.

Modeling the Clinical Variables Using Top DNA Methylation PCs

Dimensionality reduction of the methylation data was performed using PCA. The top PCs were taken as features of each sample to model each of the clinical variables in a classification setting. Specifically, for each clinical variable, we mapped their values to binary class labels using the criteria listed in Table 2. When considering each clinical variable, samples with missing values were omitted. We then constructed logistic regression models with L2 regularization using the Python scikit-learn package (version 0.20.3) following a 10-fold cross-validation procedure. In this procedure, the whole set of samples was randomly divided into 10 subsets, and each time 9 subsets were used to construct a model while the remaining subset was used to evaluate the model performance, quantified by AUROC. The 10 sets of results were then reported separately, together with their mean values. We also tried two other modeling methods, namely support vector classifier with a radial-basis kernel and random forest, and obtained largely comparable results as the logistic regression models (Table 3). This same procedure was also used when we modeled eGFR using sex, age and smoking status alone and with the top PCs.

Single-Site Epigenome-Wide Association Study (EWAS)

Baseline eGFR was calculated using the CKD-EPI equation. eGFR slope was calculated using a linear mixed model where log-transformed eGFR was used as the dependent variable, and slope was expressed as change of eGFR per year. To adjust for cell heterogeneity of whole-blood samples, cell type compositions were estimated using a reference-based approach. Using raw methylation data as input, we generated estimated cell counts for CD4⁺ T cells, CD8⁺ T cells, NK cells, B cells, monocytes, and granulocytes, using the estimate Cell Counts function implemented in the minfi package (version 1.28.4). Then for each CpG site, a linear model was constructed using either baseline eGFR or eGFR slope as the dependent variable and the methylation level (quantified by a beta value) as the independent variable. Sex, age, smoking status, duration of diabetes, hemoglobin A1c, blood pressure, experiment batch and the cell type composition estimations were also added as additional independent variables for models that allowed covariates. The p-value of each CpG site was calculated based on the null hypothesis that it had a zero coefficient in its linear model. The Bonferroni procedure was used to perform multiple hypothesis testing correction of the raw p-values. In addition, the Benjamini-Hochberg procedure was used to identify significant sites at a given false discovery rate.

In addition to using beta values to quantify methylation levels, we also tried using M values (where M=log β/(1−β)) and the results were highly similar to those based on beta values, with their corresponding CpG site p-values having a Pearson correlation of 0.967 and 0.956 for the baseline eGFR models and eGFR slope models, respectively. The corresponding Spearman correlations are 0.928 and 0.927 for baseline eGFR and eGFR slope, respectively.

Details of the Procedure for Learning the Multi-Site Models

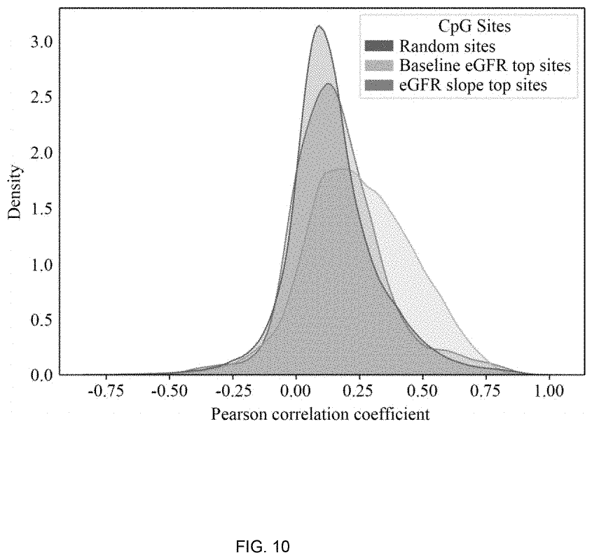

We used a multi-step procedure with nested cross-validation to perform model learning, hyper-parameter tuning, and unbiased model evaluations (FIG. 10). As a data pre-processing step, the methylation levels of each CpG site and the values of each covariate were individually standardized to have zero mean and unit variance.

In our multi-step procedure, we first randomly split the 1,268 samples into training (90%) and testing (10%) sets. Using the samples in the training set, we used the 10-fold cross-validation procedure to construct linear regression models with LASSO. The value of the regularization parameter α was chosen using grid search based on a nested 5-fold cross-validation within each training fold. The value of α chosen (denoted as α*) for each of the 10 outer training folds was determined using the following criterion:

α*=max{αϵD|Rₒ²≥max(R²)−SD(R²)}, (3)

where R² is the R² of the LASSO model using parameter α, max(R²) and SD(R²) are the maximum and standard deviation of R² among all the models with different values of α in the set D considered during the grid search. This criterion aims at finding the largest value of α that still gives a model performance close to the one with maximal R². The goal of choosing a large value of α is to ensure that only a small set of the most important CpG sites is selected from each model. Using this selected value of α, a model was trained with all the samples in the outer training fold. The model was then applied to the samples in the outer testing fold to compute the performance measures. After doing these for all the 10 outer training folds, 10 sets of performance measures were produced. This whole procedure was further repeated 10 times with different random splits of data into 10 folds each time, leading to a total of 100 models and correspondingly 100 sets of performance measures.

To produce a single model based on these 100 sets of results, we assigned a weight to each CpG site based on the number of times that it was included in the models and the performance of these models, using the following formula:

$${w}_{k} = {\sum }_{j = 1}^{10} {\sum }_{i = 1}^{10} {ρ}_{ij}^{′} \qquad ( 4 )$$

$${ρ}_{ij}^{′} = \{ \begin{matrix}{ρ}_{\text{?}} , & if {CpG}_{k} ∈ {S}_{ij} \\ 0 , & otherwise\end{matrix} \qquad ( 5 )$$

$$\text{?} \text{indicates text missing or illegible when filed}$$

where wₖ is the weight of the k-th CpG site, ρᵢⱼ is the Pearson correlation between prediction and actual values in the i-th outer testing fold for the j-th repeat, and Sᵢⱼ is the set of CpG sites selected by the i-th outer training fold for the j-th repeat with a non-zero coefficient. Based on this formula, a CpG site would generally get a higher weight if it has a non-zero coefficient in more models and/or in models that have better performance in terms of Pearson correlation.

All the CpG sites were then sorted in descending order according to their weights. A second series of linear regression models with LASSO were then constructed using different numbers of CpG sites with the largest weights as features with all samples in the original training set for training. The final number of CpG sites to use, n* was determined using the following formula that involves the Bayesian Information Criterion:

n*=max{n|BICₙ≤max(BIC)−0.1SD(BIC)}, (6)

where BICₙ is the BIC of the model involving the n highest-weight CpG sites as features, and max(BIC) and SD(BIC) are the maximum and standard deviation of BIC among all the models with different number of CpG sites, respectively. This formula aims at maximizing the number of CpG sites while having a model with a BIC close to the one with the minimal BIC. This time, the number of CpG sites is to be maximized because the highest-weight CpG sites should already be the most important ones, and including more of them in the model can ensure its robustness. The performance of the model that involved the n* highest-weight CpG sites was then evaluated objectively using the original testing set, which was not involved in any training and parameter tuning steps described above.

Finally, all 1,268 samples were used together to train a final model for baseline eGFR and another model for eGFR slope, both using the same procedure described above to determine the number of CpG sites. Then with these chosen CpG sites, we also trained another version of these two models without including the covariates. Since these final models involved all 1,268 samples in model training and parameter tuning, there were no left-out samples in the primary cohort that could objectively evaluate their performance.

Functional Significance of Our CpG Sites' Methylation Levels in Kidney Samples

Seven CpG sites were selected to check their methylation levels in kidney samples using a published data set with methylation data from 506 human kidneys. In this data set, the samples belong to five groups based on the donors' disease status, namely Con (normal kidneys, 113 samples), CKD (eGFR<60, 101 samples), DKD (having both CKD and diabetes, 63 samples), DM (having diabetes but not CKD, 97 samples), and HTN (having hypertension but not CKD, 132 samples).

Among the seven CpG sites selected for lookup, one (cg21573651) was associated with both baseline eGFR and eGFR slope in the single-site analysis. The other six CpG sites (cg17944885, cg06449934, cg02304370, cg21919729, cg04610187 and cg18593194) were associated with baseline eGFR and were the top six sites among the 36 CpG sites identified in both single-site and multi-site analyses.

Validation of the Models in the Pima Indian Cohort

The Pima Indian cohort contained 327 participants with DKD. Baseline eGFR, eGFR during subsequent follow-up and other clinical variables were measured for each participant. DNA methylation was measured by Illumina Infinium HumanMethylation450K Beadchip.

To use this cohort to evaluate the performance of models constructed from the primary cohort, we took the intersection of CpG sites passing quality control in the two cohorts. All samples in the primary cohort were then used to learn the baseline eGFR and eGFR slope models with these CpG sites provided for selection only, using the same procedure as described before. These models were then applied to the Pima Indian cohort for comparing the predicted baseline eGFR/eGFR slope values and their corresponding actual measurements.

Risk Equations Comparison

To calculate the eGFR of each subject five years after the baseline measurements using the eGFR slope determined by Equation 1 and 2, the following formula is used:

$${c}_{i} = {β}_{1} + {b}_{1 i} = log ( \frac{{( eGFR slope )}_{i}}{100} + 1 ) , \qquad ( 7 )$$

$${( eGFR )}_{i 5} = {( eGFR )}_{i 0} \times {e}^{5 {c}_{i}} , \qquad ( 8 )$$

where (eGFR)ᵢ₀ and (eGFR)ᵢ₅ are the eGFR of i-th individual at baseline and five years after the baseline, respectively. We defined subject i to have ESKD in five years after the baseline if (eGFR)ᵢ₅<15 ml/min/1.73 m².

For each patient, the actual ESKD status was determined using the above method based on his/her actual eGFR slope obtained by making use of all his/her eGFR measurements during the follow-up period. Similarly, the ESKD status predicted by our model was produced using the above method based on the predicted eGFR slope, the multi-site model of which was constructed using DNA methylation. This was achieved by a 5-fold cross-validation procedure, in which every time 4/5 of the patients were used to train the multi-site model, which was applied to the remaining 1/5 of the patients to predict their 5-year ESKD status. The risk scores of the risk equations for renal outcomes by JADE risk model and UKPDS-OM2 were calculated following the descriptions in the original publications.

An independent nested case-control cohort of 181 individuals with type 2 diabetes, of which 80 developed ESKD during follow-up, were included to examine association between blood methylation level and progression to ESKD.

Results

Genome-Wide DNA Methylation Trends are Associated with Baseline Kidney Function

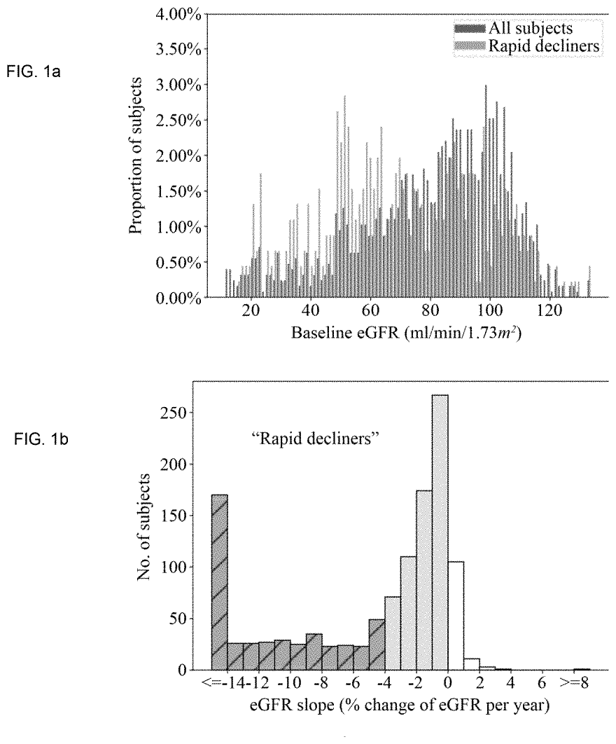

Blood samples of 1,271 patients with type 2 diabetes from the Hong Kong Diabetes Register (HKDR) were collected at baseline. Among all patients, 19.7% had DKD at baseline, defined as having an estimated glomerular filtration rate (eGFR)<60 ml/min/1.73 m², and all patients were free of pre-existing cardiovascular complications (Table 3). The samples were selected using a nested case-control design, whereby each subject free of DKD at follow-up was matched with a case of incident DKD. During a median follow-up period of 14.6 (Q1-Q3: 8.3-19.4) years (censored on Jun. 30, 2017), 33% developed end-stage renal disease (ESRD). During the follow-up period, the included subjects had a median number of eGFR measurements of 29 (Q1-Q3: 15-46), and the mean eGFR slope during follow-up was −5.55% change of eGFR per year (Materials and Methods, FIGS. 1a-1b).

Genome-wide DNA methylation levels were measured from each sample using Illumina Infinium Human Methylation450K Beadchip according to the standard workflow, followed by standard data processing (Materials and Methods). After filtering and normalization, 434,908 CpG sites and 1,268 samples were retained, with the methylation level of each site in each sample quantified by a beta value. Following some previous studies, all CpG sites on the sex chromosomes were omitted.

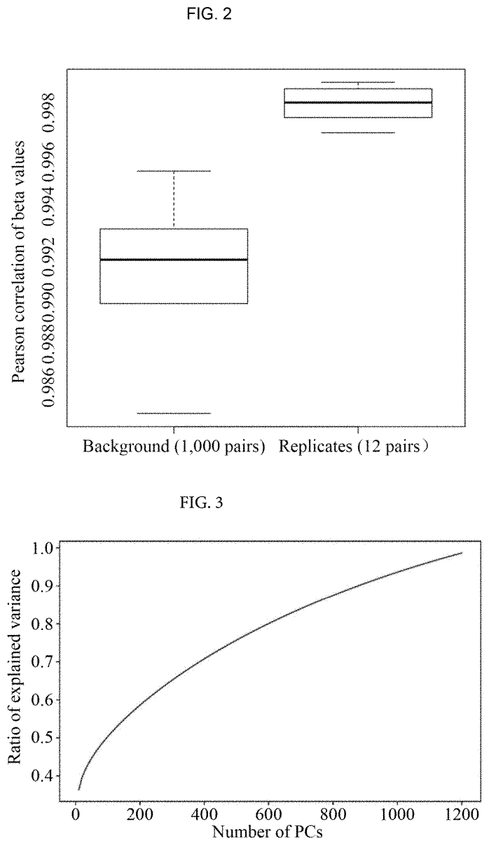

For 12 patients, methylation levels were measured independently from 2 technical replicates. Beta values among replicate samples had a median Pearson correlation of 0.998 and these correlation values were significantly higher than those among random sample pairs (FIG. 2; p=2.51×10⁻⁹, two-sided Wilcoxon rank-sum test), indicating high reproducibility of the data.

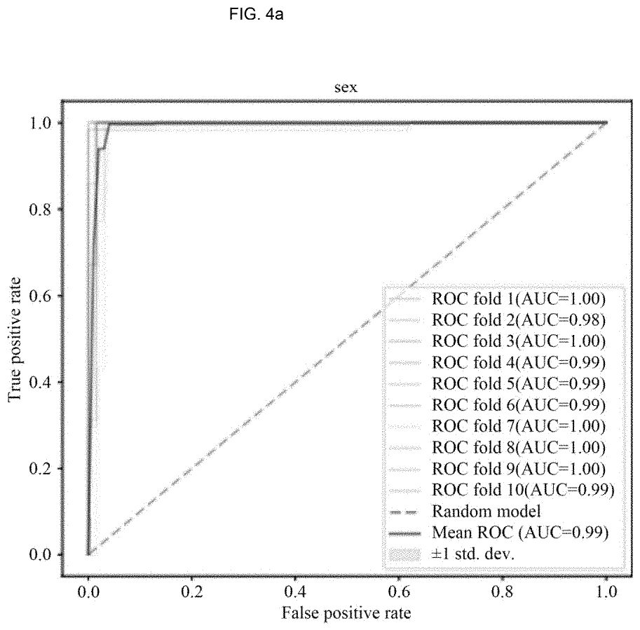

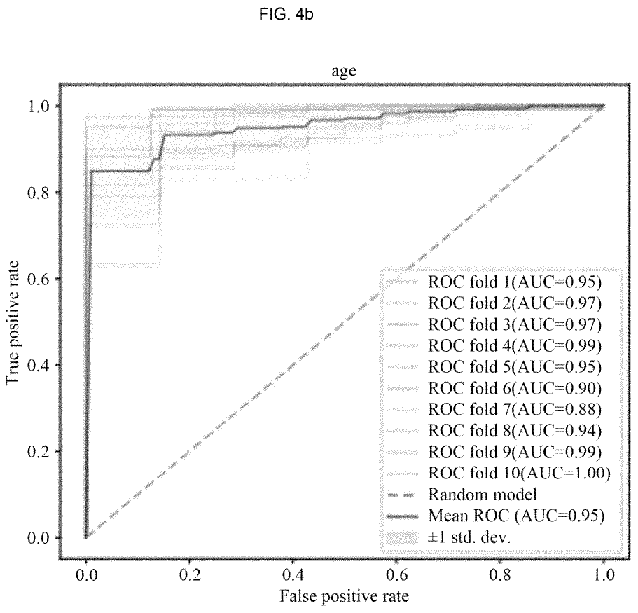

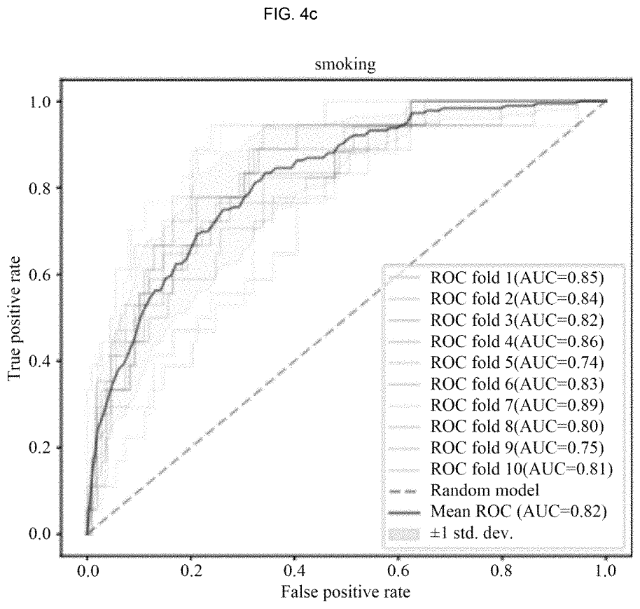

To investigate whether global DNA methylation trends are associated with clinical variables, we performed principal component analysis (PCA) of the methylation data. Using the top 50 principal components (PCs), which explained 45% of the total data variance (FIG. 3), as features, we constructed a regularized logistic regression model for each clinical variable as the target trait in turn using a 10-fold cross-validation procedure, which trained the model and evaluated its performance on mutually exclusive subsets of samples (Material and Methods). The models with highest cross-validation performance were those for sex (mean area under the receiver-operator characteristics [AUROC] of the 10 testing sets=0.99), age (mean AUROC=0.95) and smoking status (mean AUROC=0.82), and these results were robust across different sets of training samples (FIGS. 4a-4c). These findings are consistent with previous reports that DNA methylation is highly associated with sex, age and smoking and they further support the quality of our methylation data.

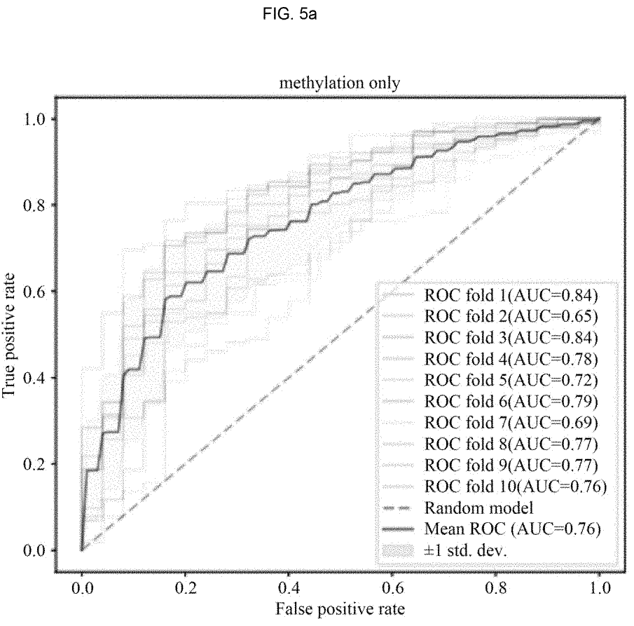

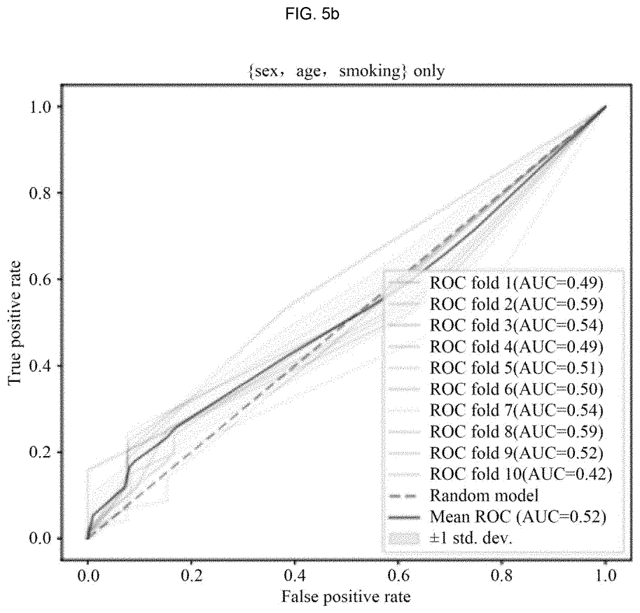

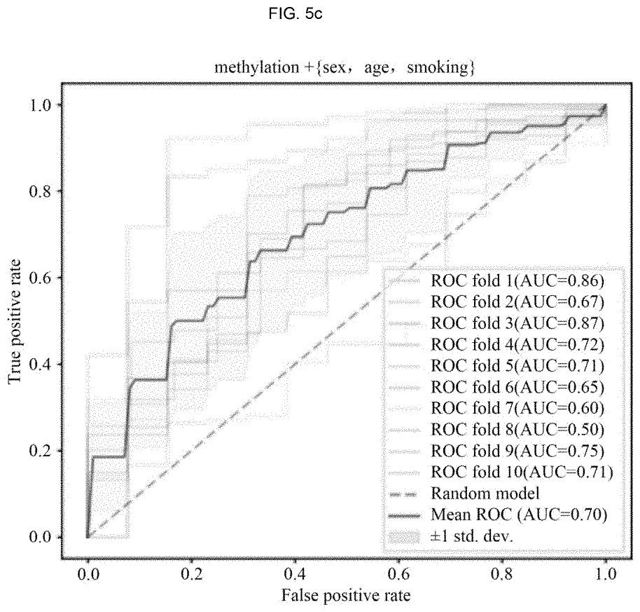

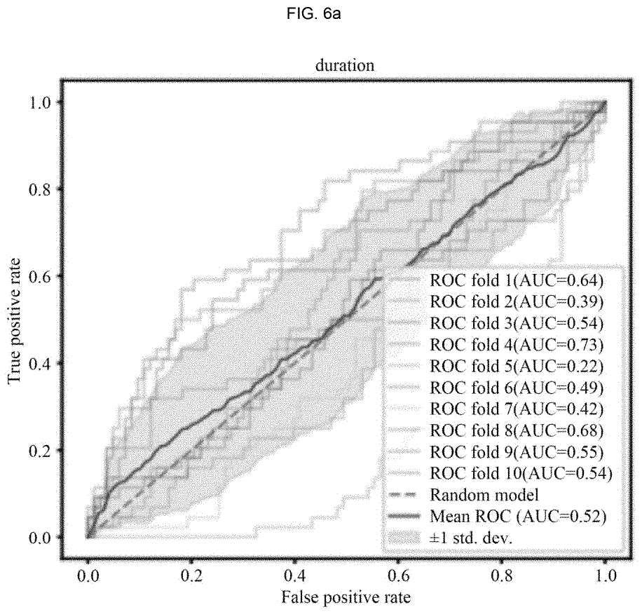

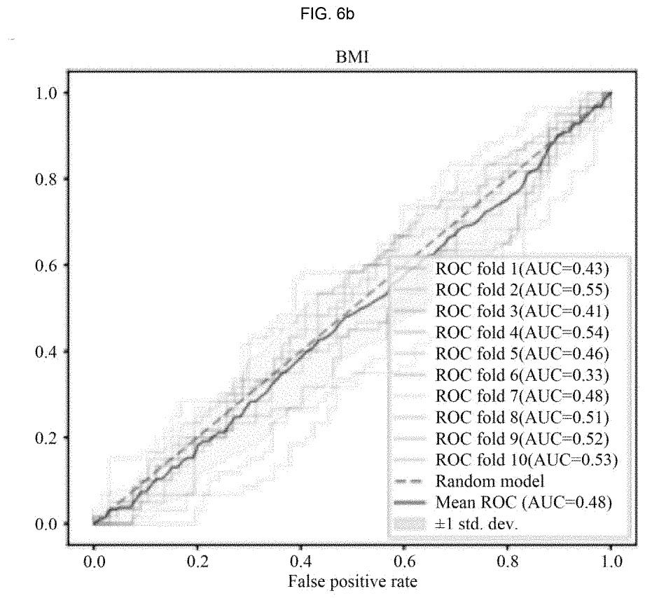

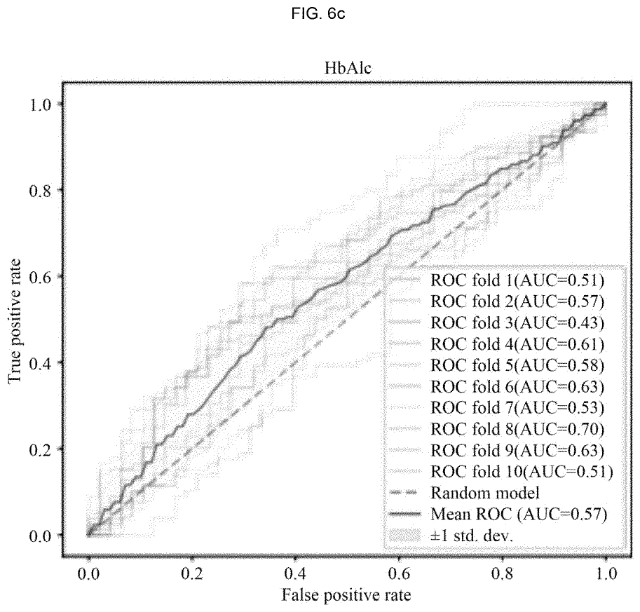

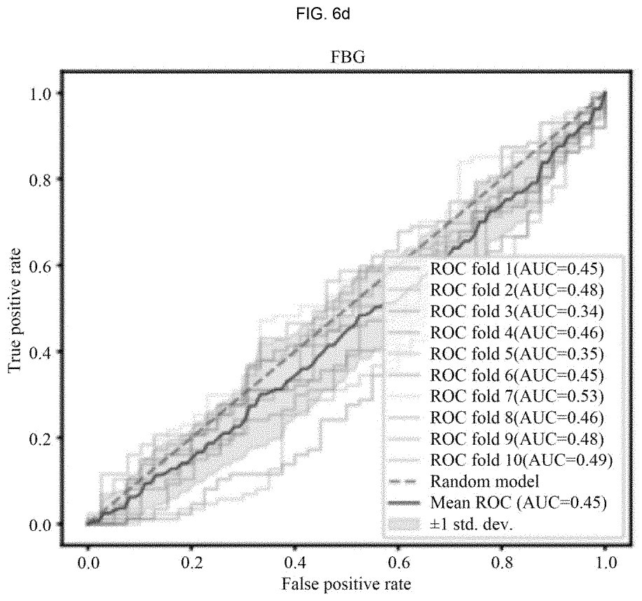

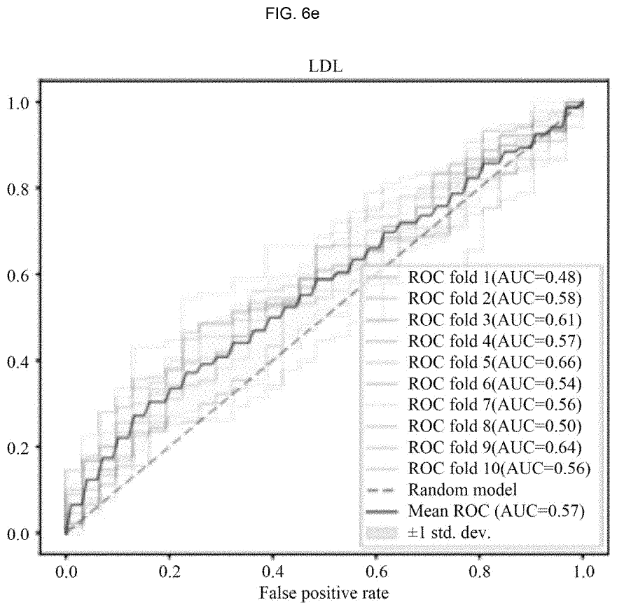

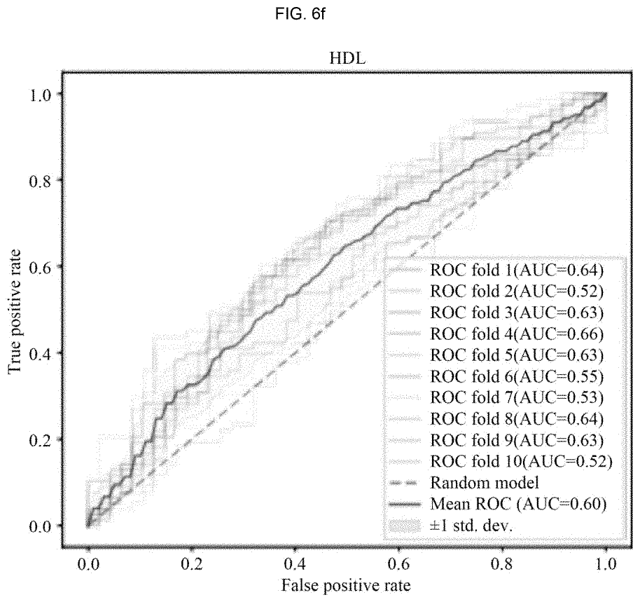

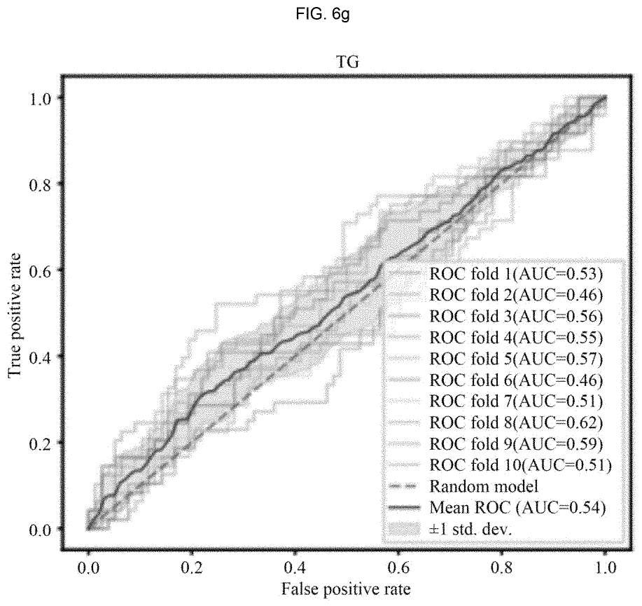

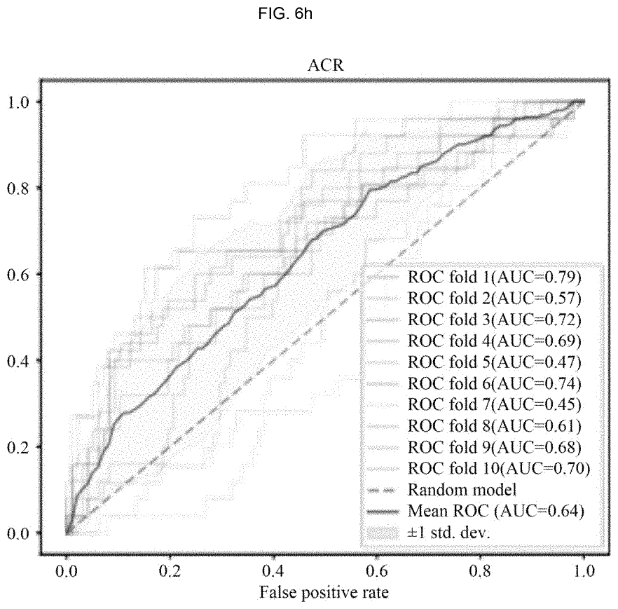

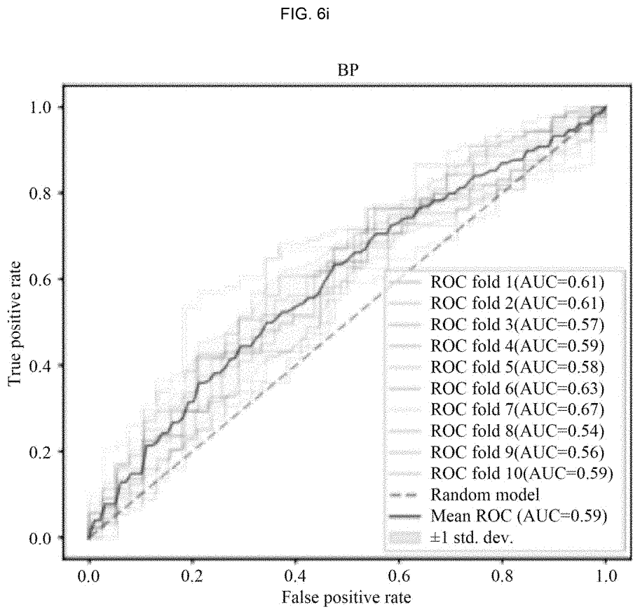

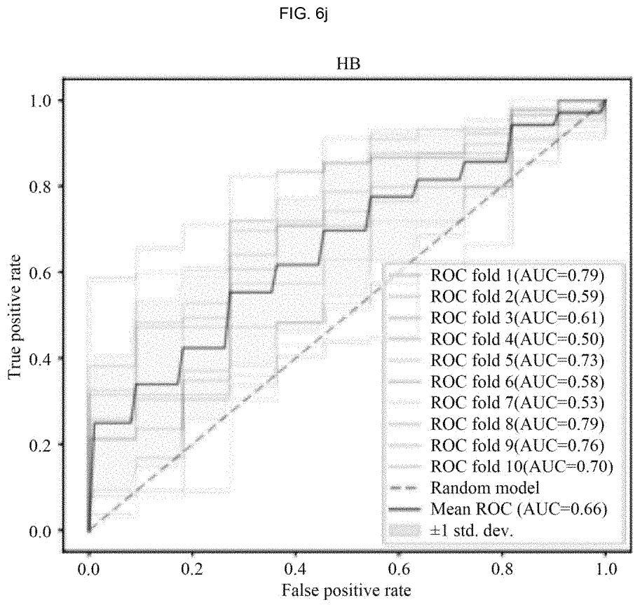

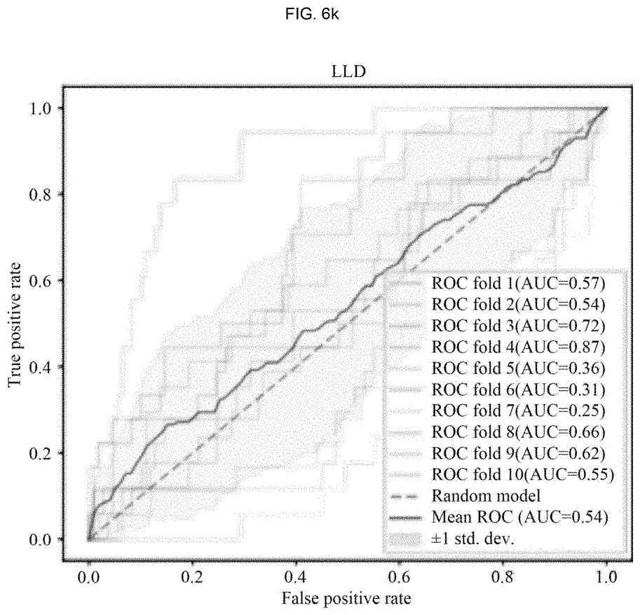

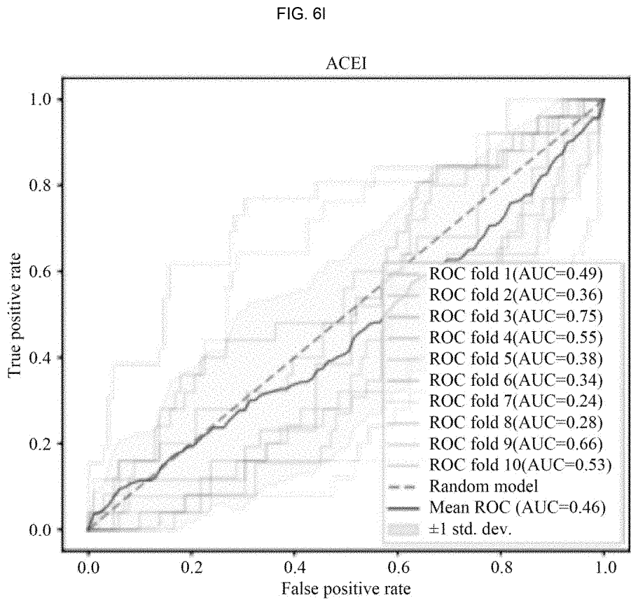

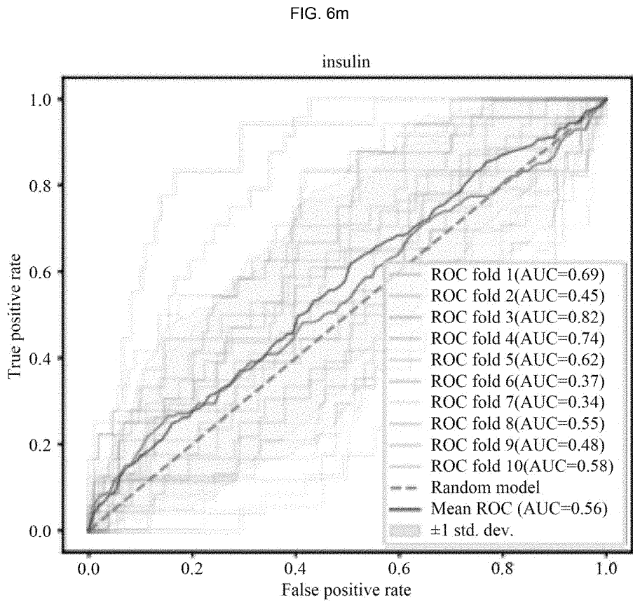

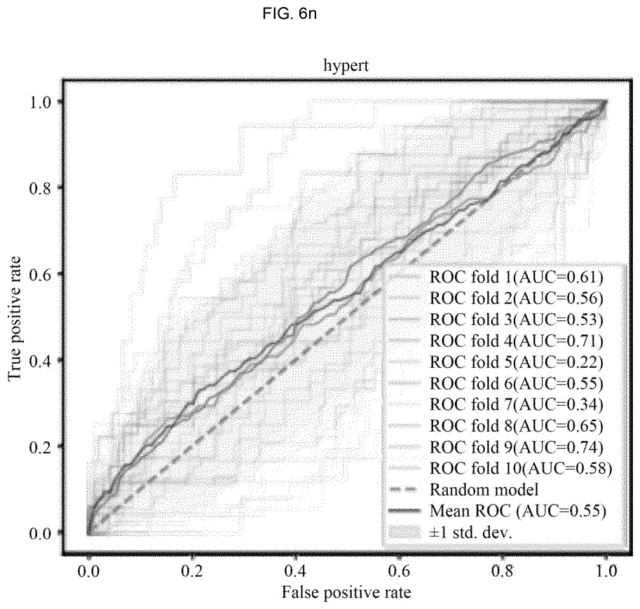

As expected, DNA methylation was associated with renal function, with the models for baseline eGFR achieving a fairly high mean AUROC of 0.76 (FIG. 5a). In contrast, most of the other clinical variables were not strongly associated with DNA methylation (FIGS. 6a-6n). To see if this association between DNA methylation and baseline eGFR was due to confounding factors caused by sex, age or smoking status, we also constructed models of baseline eGFR using these three variables alone, and found that the AUROC values were close to the expected value of 0.5 for a random model (FIG. 5b), showing that baseline eGFR could not be inferred by these variables. Furthermore, we constructed models using both the 50 top PCs of DNA methylation and these three variables as features together, and found the resulting AUROC values not higher than the ones having the 50 PCs alone (FIG. 5c). Together, these results show that there is a fairly strong association between baseline eGFR and global methylation trends independent of the other clinical variables strongly correlated with DNA methylation.

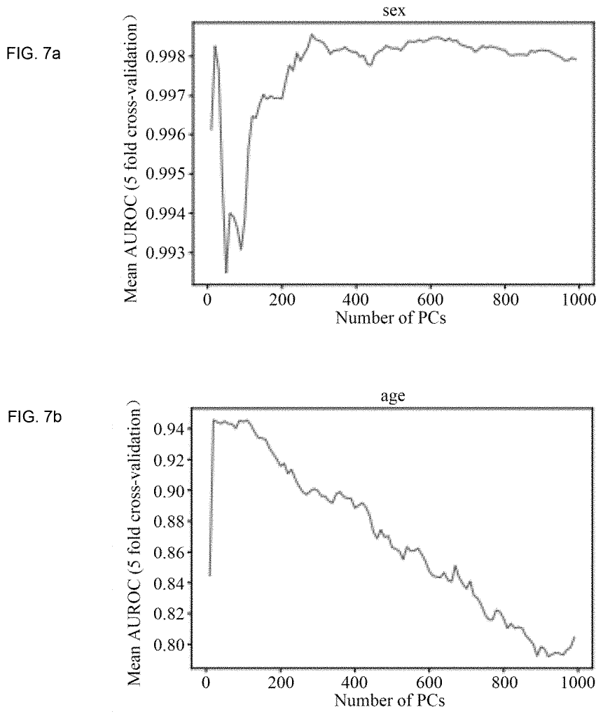

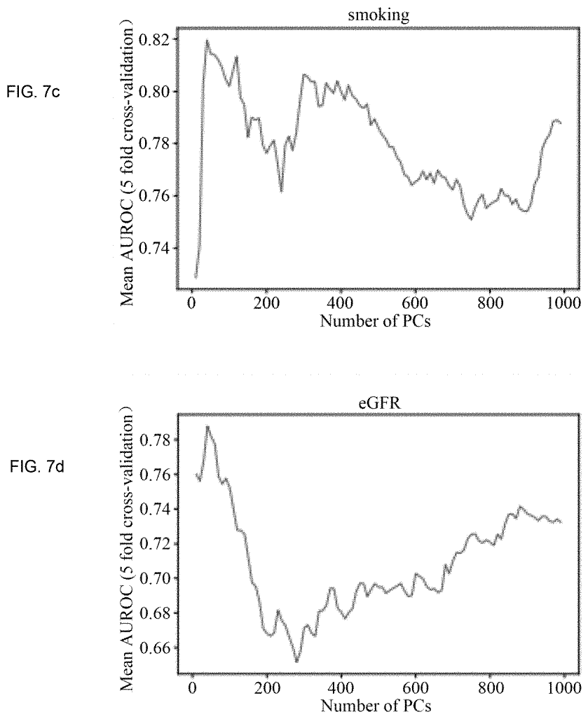

We repeated the modeling procedures using other numbers of top methylation PCs as features (FIGS. 7a-7d). For the models for baseline eGFR, similar to those for age and smoking status, the mean AUROC value generally displayed a decreasing trend as more PCs were included, showing that the most accurate models could be obtained by considering only a small number of the most informative features. Based on this finding, we next examined the associations of the methylation levels of individual CpG sites with renal function.

Methylation Levels of Individual CpG Sites are Associated with Baseline Renal Function and Renal Function Decline

To find out individual CpG sites associated with renal function, we performed an epigenome-wide association study (EWAS) of baseline eGFR. In addition to setting baseline eGFR as the target trait, since some recent studies have reported that CpG methylation levels are predictive of the decline of eGFR overtime, we also set eGFR slope as an additional target trait (Materials and Methods). We included sex, age, smoking status, duration of diabetes, hemoglobin A1c, blood pressure, experiment batch and cell type composition estimations as covariates, and used the methylation level of each CpG site as an independent variable to form a linear model of each target trait. A corresponding p-value was then computed for each site based on the null hypothesis that the coefficient of it in the model was zero.

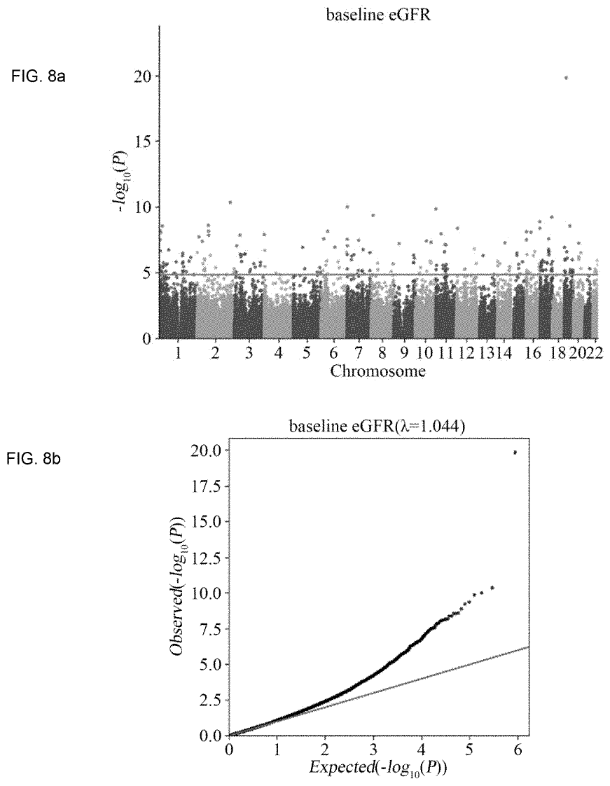

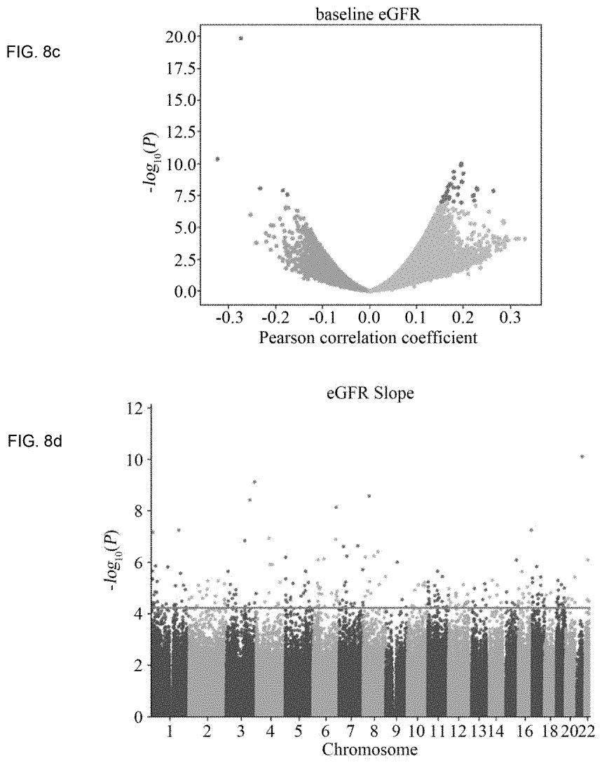

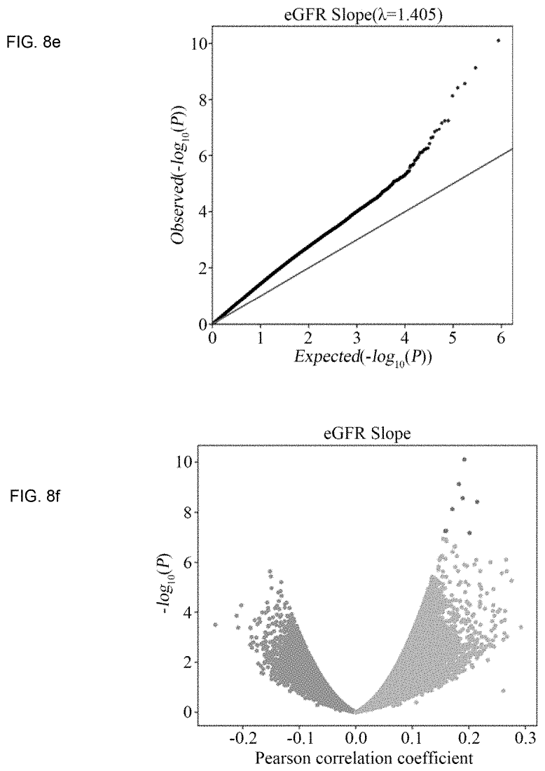

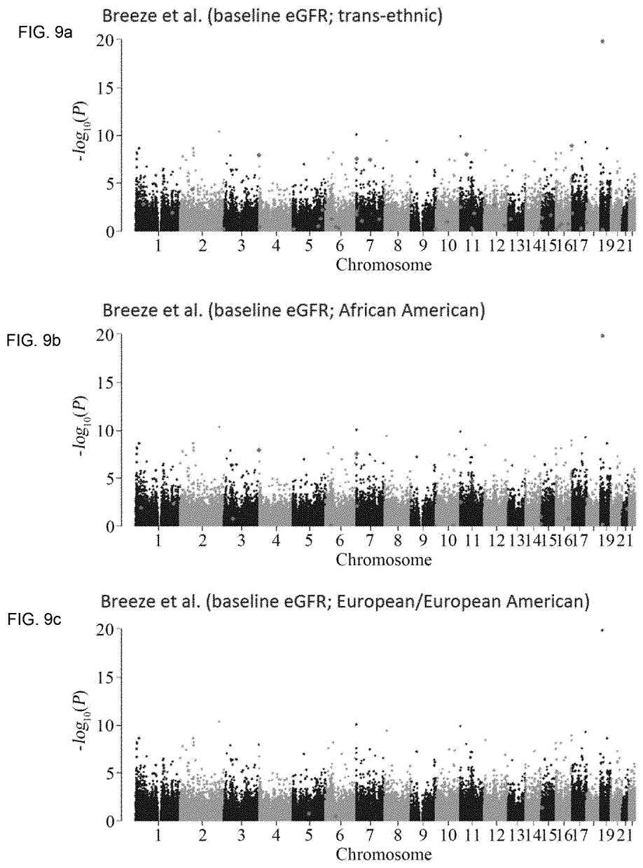

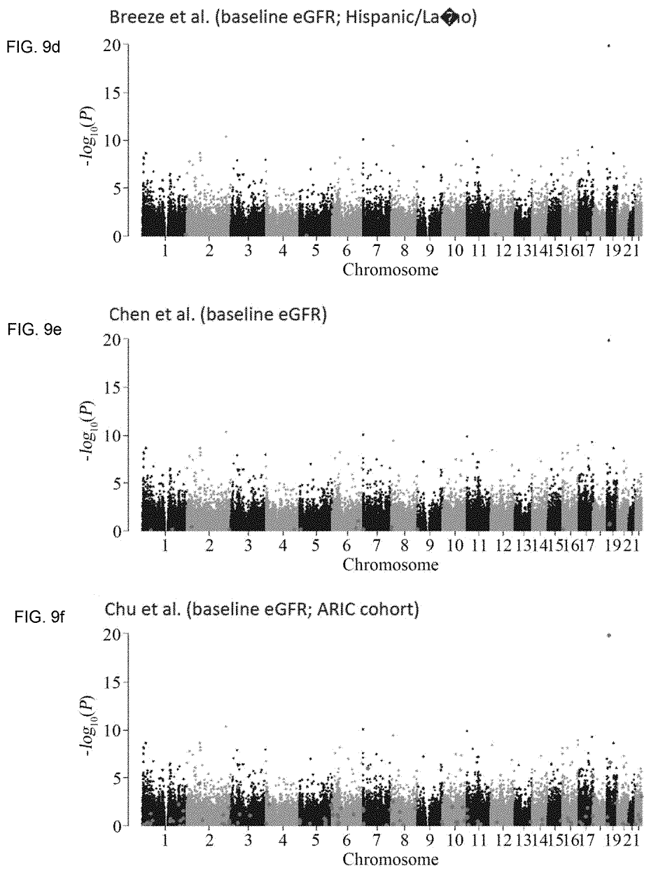

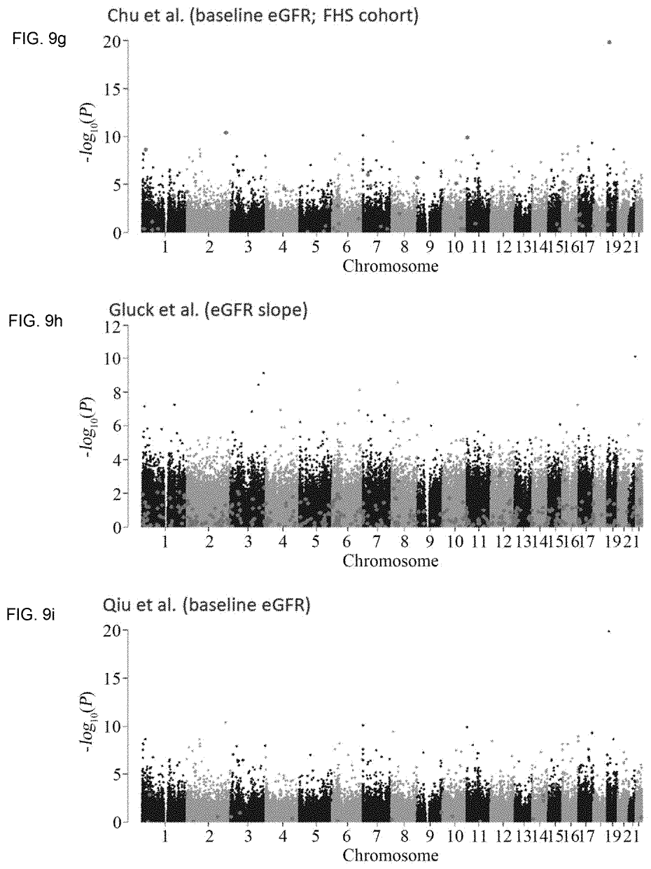

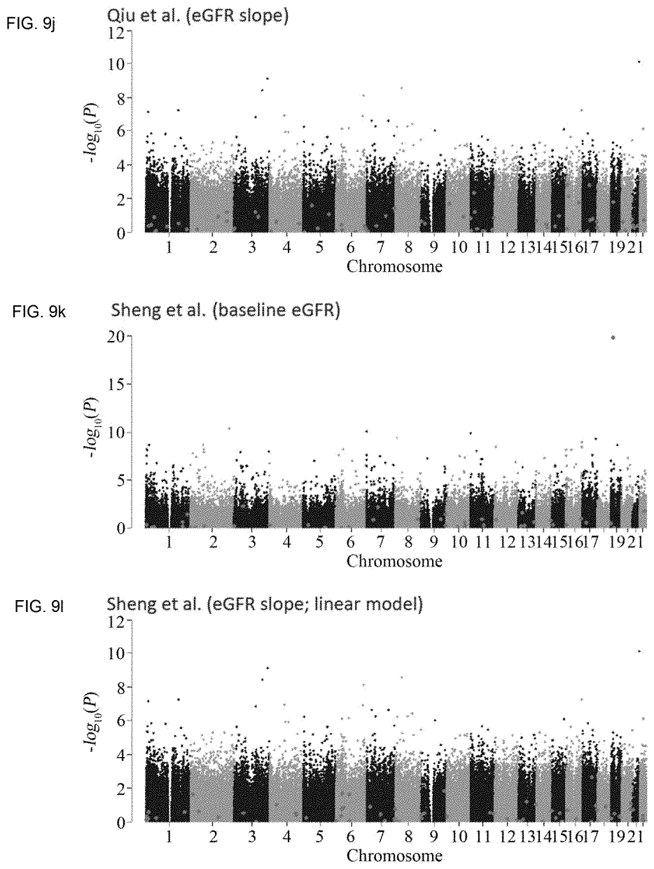

For baseline eGFR, 40 CpG sites reached epigenome-wide significance by having a Bonferroni-corrected p-value below 0.05, and 386 CpG sites were statistically significant at false discovery rate (FDR)=0.05 (FIGS. 8a-8c, Table 4). The most significant CpG site, cg17944885 (Bonferroni-corrected p=5.16×10⁻¹¹), located between ZNF788 and ZNF20 on chromosome 19, was also reported in several previous studies to have its DNA methylation level associated with renal function in various populations (FIGS. 9a-9l). In general, our results are most consistent with those reported in Chu et al. based on their data from the ARIC and FHS cohorts and Breeze et al. based on data from multiple studies and ethnicities, with a number of their reported top sites having association p-values clearly separated from the background in our data, even though none of these previous studies were based on Chinese-specific cohorts or population with only patients with type 2 diabetes (FIG. 10). For example, other than cg17944885, 13 significant CpG sites at FDR=0.05 in our cohort, including cg25364972, cg02304370, cg12065228, cg21745599, cg16292343, cg05554494, cg22386583, cg09299075, cg13924998, cg07814567, cg03919650, cg19942083, and cg26099045 were also reported as significant signals in either ARIC or FHS cohort, and one significant CpG site in our data, cg23597162, was identified in both the ARIC and FHS cohorts. Interestingly, four of the sites with a Bonferroni-corrected p-value below 0.05 (cg04983687, cg23845009, cg01676795, cg22460173) and one other significant site at FDR=0.05 (cg26099045) in our cohort were also reported as significant in a recent meta-analysis, but they were not reported in earlier studies of individual cohorts, suggesting that these trans-ethnic signals may be stronger in our Chinese cohort and thus in other populations they were identified only when a larger sample size was achieved by the meta-analysis.

In order to identify methylation sites that may be informative for predicting decline in renal function, association between baseline methylation status and subsequent eGFR slope was examined. Eight CpG sites had a Bonferroni-corrected p-value below 0.05 and 74 CpG sites were significant at FDR=0.05 (FIGS. 8d-8f, Table 4). The most significant CpG site is cg10272901 (Bonferroni-corrected p=3.41×10⁻⁵), located in a CpG island on chromosome 21. None of these 82 sites was reported as significantly associated with eGFR slope in several related studies, conducted mainly in the general population rather than population with diabetes. When we performed reciprocal lookup of the previously reported top sites from our data, we found several sites reported by Gluck et al., identified based on data from multiple populations, to have marginally significant association p-values in our data (FIGS. 9a-9l), including cg15826891 (p=5.29×10⁻⁵ in our data), which is located within the MIR100HG non-coding gene locus on chromosome 11 and cg02950701 (p=1.26×10-4 in our data), which is located within the protein-coding gene CCNY locus on chromosome 10.

These results confirm that methylation levels of individual CpG sites are also associated with both baseline renal function and the decline of renal function overtime in a Chinese population with type 2 diabetes, as have been previously shown in some other populations. Some specific signals (such as methylation level at cg17944885) appear to have consistently significant association with baseline renal function across various populations. Our analysis also discovered a large number of novel sites with significant associations not reported before.

A Multi-Site Approach to Identifying Sets of CpG Sites Indicative of Renal Function

The single-site approach described above, though commonly used in the literature, has two important limitations. First, some CpG sites that are not strongly associated with renal function by themselves could actually complement other sites by explaining some important residual renal function differences. These “auxiliary” sites cannot be identified by the single-site approach. Second, some significant CpG sites identified by the single-site approach could be strongly correlated with each other (FIG. 10), due to spatial dependency or other reasons, leading to redundancy and a possibility of diverting the attention to some non-functional sites.

To tackle these limitations, we developed a multi-site approach that considered all CpG sites at the same time and selected a subset of them that together can best model base line eGFR/eGFR slope (Materials and Methods). Briefly, we used LASSO (least absolute shrinkage and selection operator) to construct regression models, which aims at fitting linear models with only a small number of CpG sites having a non-zero coefficient. Performance of each model was evaluated using cross-validation, while the final set of CpG sites was selected using a nested procedure that involves the Bayesian Information Criterion (BIC) to balance between model complexity and performance. The constructed models were finally evaluated using left-out testing sets not involved in either training the models or tuning the hyper-parameters.

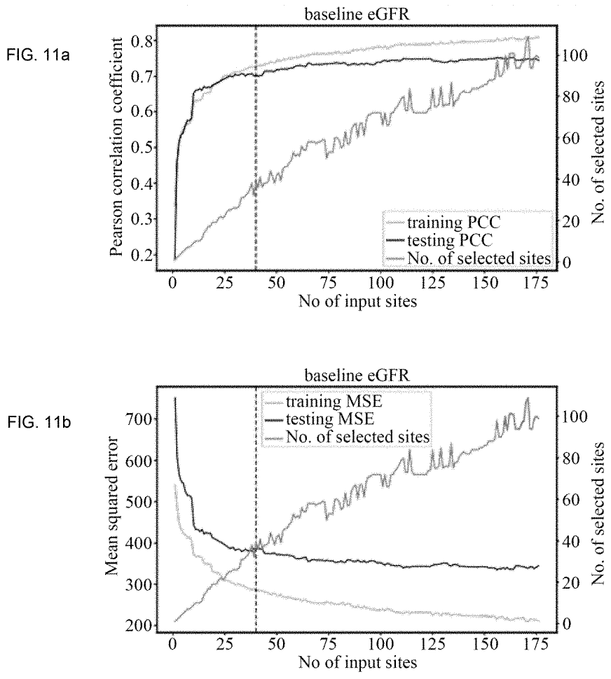

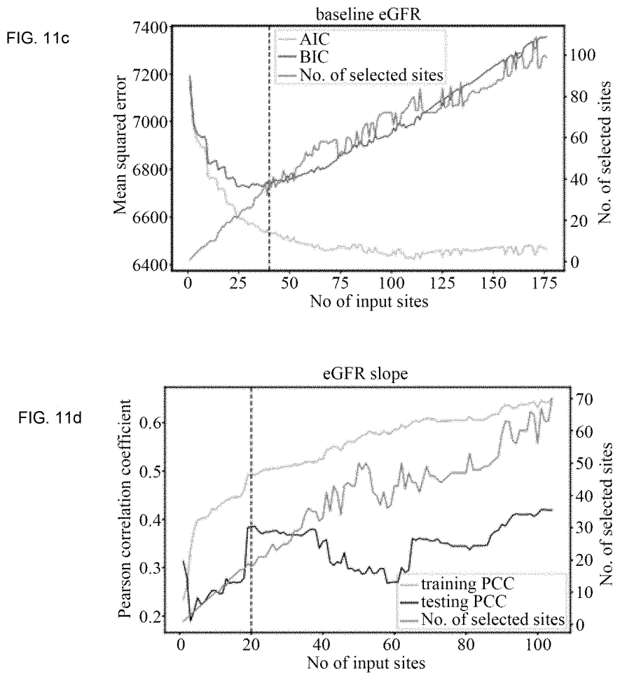

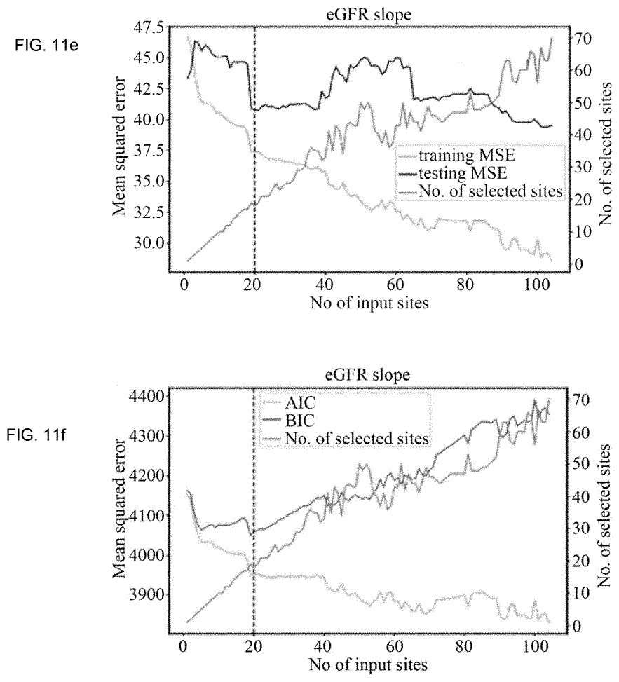

FIGS. 11a-11f show the performance of the models at different feature selection thresholds as evaluated by the overall testing set. In general, when a less stringent feature selection threshold was used, more CpG sites would be included in the models and the training performance would be higher, yet the performance on the left-out testing sets was not necessarily better, which indicates that overfitting could have occurred when the models contained too many CpG sites. This observation confirms the importance of evaluating the models using data not involved in model training. For both baseline eGFR and eGFR slope, the maximal modeling performance, as judged by both the Pearson correlation between the actual and inferred values or their mean squared error computed from the left-out testing data, could be achieved with a stringent feature selection threshold and a corresponding small number of CpG sites included, which is consistent with the PCA results described above.

Considering both the model performance and the complexity of the models, our BIC-based procedure automatically determined the feature selection thresholds. According to the left-out testing data not involved in this procedure, at these selected thresholds, the Pearson correlation between the actual baseline eGFR values and the values inferred by the models was 0.704, and it was 0.386 for eGFR slope (FIGS. 11a, 11d).

The Multi-Site Models Capture Relationships Between DNA Methylation and Renal Function in Multiple Populations

After confirming the validity of our procedure, we next used it to rebuild the models using the whole set of samples. In these “final” models, 64 and 37 CpG sites were included in the case of baseline eGFR and eGFR slope, respectively (Tables 5, 6).

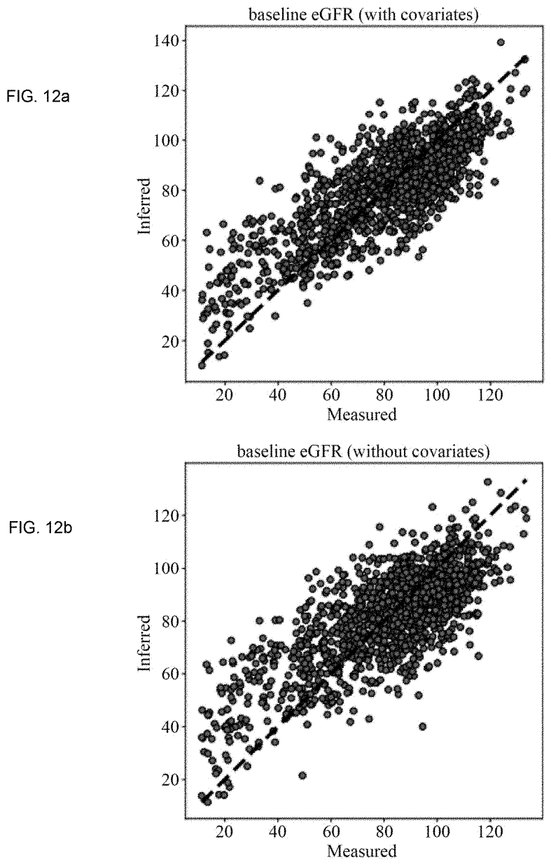

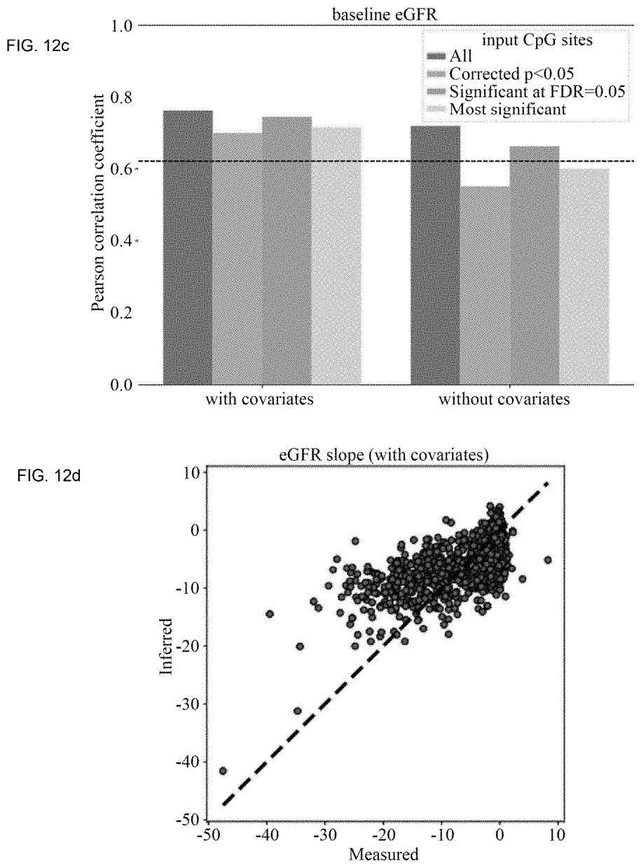

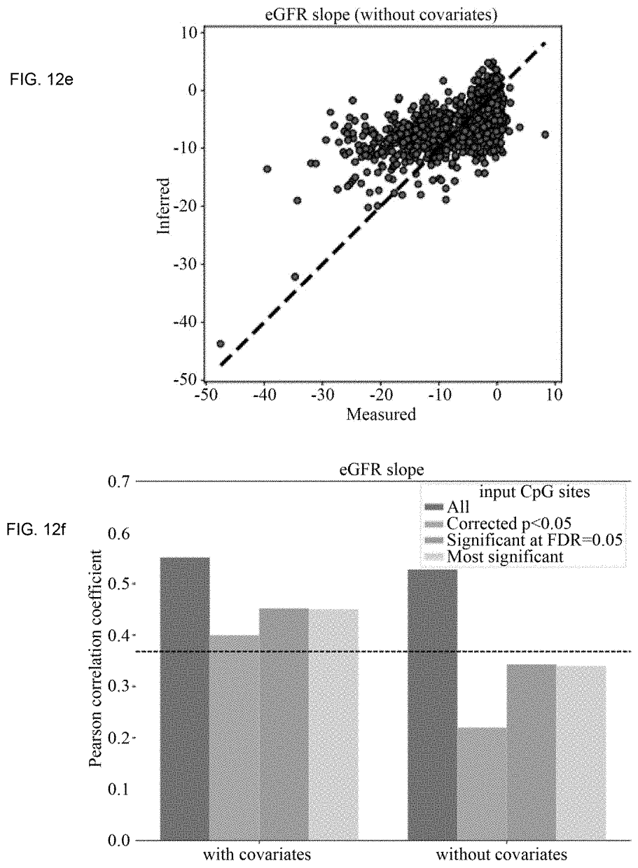

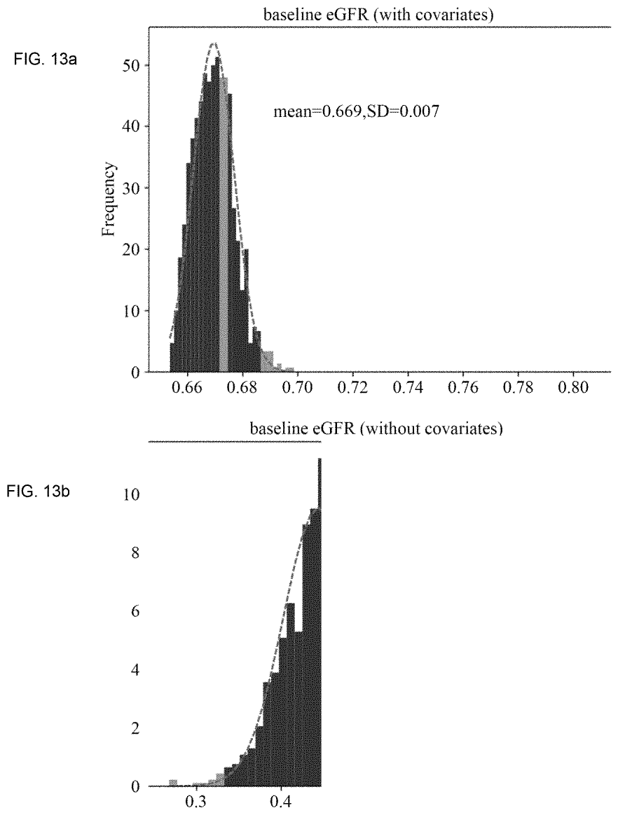

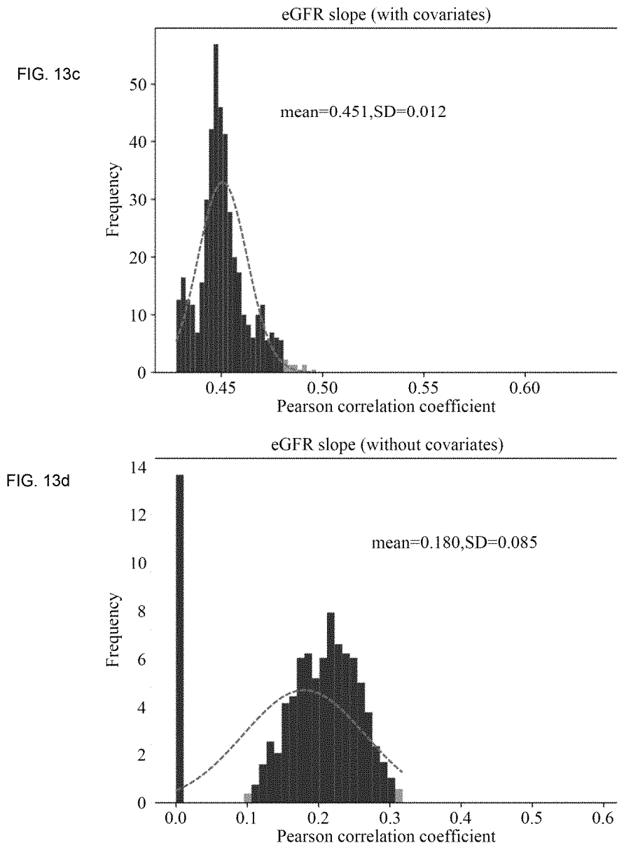

For baseline eGFR and eGFR slope, the actual values and the values inferred by our final models had Pearson correlations of 0.806 and 0.635, respectively (Table 7 and FIGS. 12a, 12d), which are substantially higher than the largest absolute Pearson correlations of single CpG sites (0.331 and 0.292 for baseline eGFR and eGFR slope, respectively, FIGS. 8c, 8f). To examine the effects of the covariates, we also used the same procedure to construct models without them. We found the modeling performance to decrease in terms of both correlations and mean squared errors when the covariates were excluded from the models (Table 7 and FIGS. 12b, 12e), which suggests that including the covariates could improve the robustness of the models by eliminating some confounding factors. We also constructed models using the same number of CpG sites randomly selected from the whole genome, and found that the real models performed substantially better than these random models (FIGS. 13a-13d).

In our final models, while some of the CpG sites included were also significantly associated with renal function in the single-site analysis, such as the most significant sites cg17944885 for baseline eGFR and cg10272901 for eGFR slope, some others did not have significant associations by themselves, showing that they were included in the multi-site models due to the extra information that they carried for inferring the target traits missed by the other CpG sites. The most significant site cg17944885 for baseline eGFR was also included in the multi-site model for eGFR slope, although it was not significant for eGFR slope in the single-site analysis. Interestingly, one of these sites for the baseline eGFR model, cg13408344, has been reported in a recent meta-analysis to be significantly associated with baseline eGFR, suggesting that our multi-site method is identifying clinically significant CpG sites that can be uncovered using larger EWAS sample sizes.

As an additional evaluation of the importance of these CpG sites that are individually not strongly associated with the target traits, we compared our final models with three alternative models constructed with different choices of input CpG sites, namely 1) the subset of sites in our final models that had a single-site Bonferroni-corrected p-value <0.05, 2) the subset of sites in our final models that were significant at FDR=0.05 in the single-site analysis, and 3) the sites with the most significant single-site p-values among all CpG sites, with the total number of sites the same as our final models (64 for baseline eGFR and 37 for eGFR slope). All these alternative models did not perform as well as our original models (FIGS. 12c, 12f, Table 8), showing that the auxiliary CpG sites played crucial roles in modeling baseline kidney function and its decline overtime.

To evaluate whether the selected sites could successfully classify people with or without renal disease, we constructed regularized logistic regression models using the above choices of CpG sites for baseline eGFR and eGFR slope. All the models performed well in these classification tasks, with sites selected by our original LASSO regression models achieving a mean AUROC of 0.893 for baseline eGFR and 0.805 for eGFR slope (Table 9), demonstrating the ability of these sites in recognizing people with potential renal dysfunction.

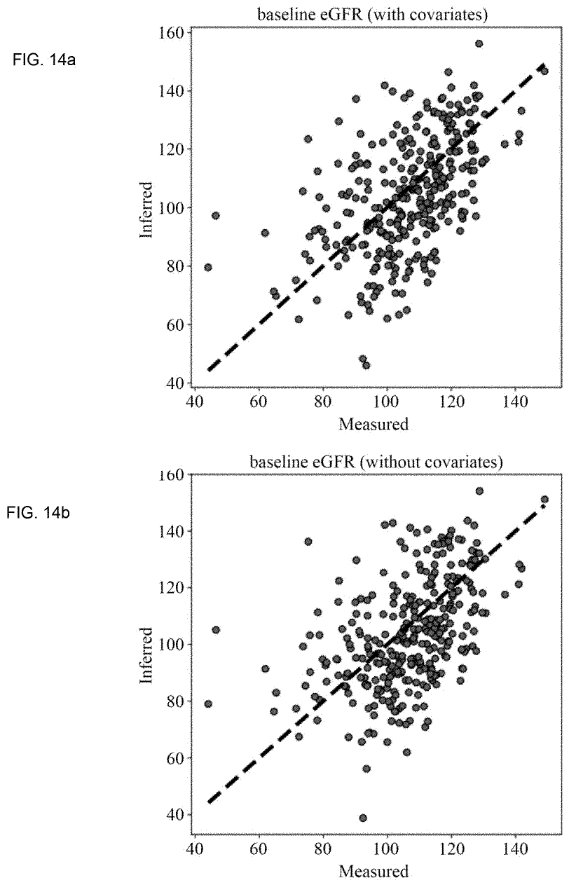

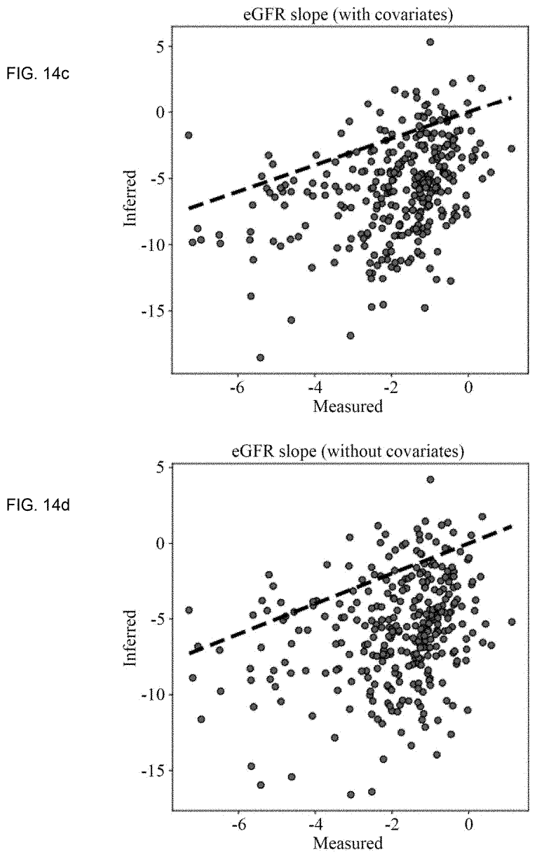

Since these final models were constructed using all samples, there were no left-out samples from our cohort for an independent evaluation of their performance. Therefore, we tested the models using a second cohort of data consisting of subjects with type 2 diabetes. This cohort involved genome-wide methylation measurements of blood samples from 327 Pima Indian subjects with type 2 diabetes. Since the CpG sites that passed the data processing procedures of the two data sets were different, we rebuilt the models using all samples in the primary cohort but considered only CpG sites that passed QC parameters in both cohorts as features. We then applied these models to thePimaIndiancohortandcomparedtheinferredbaselineeGFRandeGFRslope values with the actual ones. In the Pima Indian cohort, the eGFR slope was determined using a linear regression for each individual and expressed as change of eGFR per year, which is different from the eGFR slope definition in the primary cohort. The results (Table 7 and FIGS. 14a-14d) show that the models also achieved good performance for predicting baseline eGFR and eGFR decline in type 2 diabetes on this set of independent data despite the difference in ethnicity of the subjects in the two cohorts. For example, when applying our model to the Pima Indian cohort, the predicted and actual baseline eGFR values had a Pearson correlation of 0.510. Similarly, for eGFR slope, when applying our model to the Pima Indian cohort, the predicted and actual baseline eGFR values had a Pearson correlation of 0.356, which is very close to the correlation value of 0.386 when we tested our procedure using a left-out testing set in the primary cohort.

Proximal Genes of the Selected Sites in the Single-Site and Multi-Site Analyses have Potential Kidney Functions

We next evaluated the functional significance of the genes proximal to (within 1 kb) the sites identified in our single-site and multi-site analyses by checking whether they have been reported as potentially related to kidney function in previous studies. We collected these potential kidney function-related genes from a number of previous studies that identified the genes using various types of data, including DNA methylation data of blood samples from people with or without kidney disease, bulk RNA expression data of human kidneys, and single-cell RNA sequencing data of mouse kidneys.

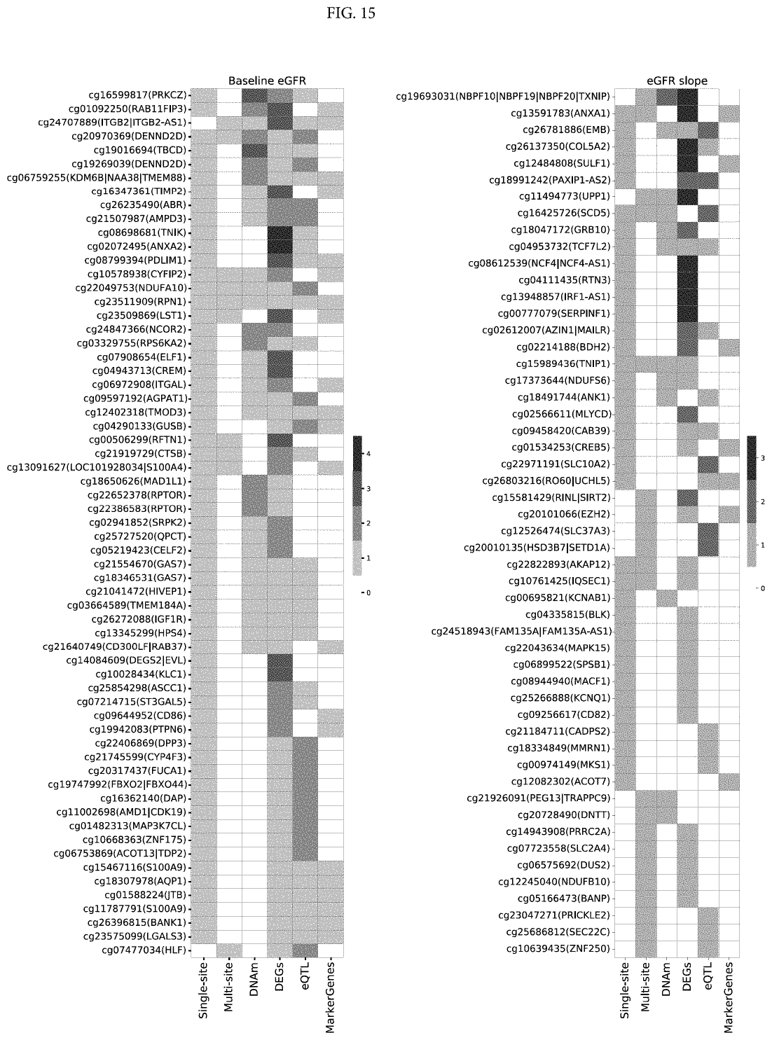

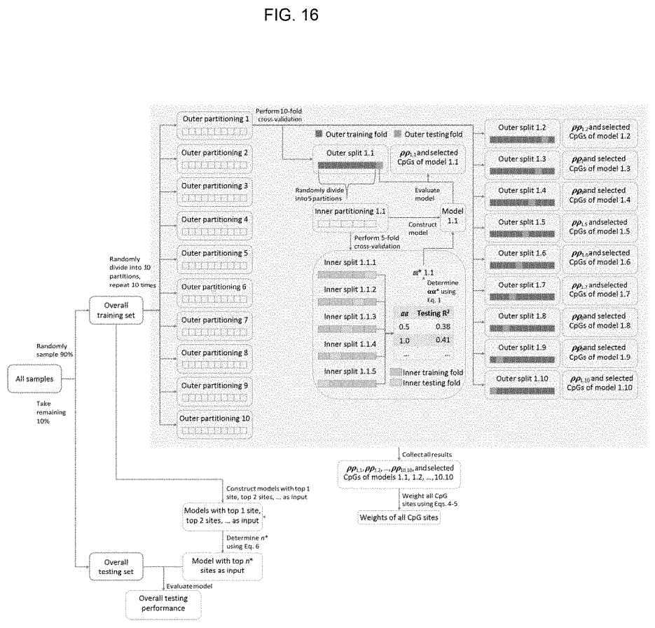

Out of the 348 CpG sites identified by our single-site and multi-site analyses as associated with baseline eGFR, 230 of them (66.1%) were reported in at least one of these previous studies (FIG. 15), which corresponds to a 1.69-fold enrichment as compared to the set of all human genes (p=2.00×10⁻²⁴, hypergeometric test).

Noticeably, the CpG site cg24707889, located in the upstream region of the ITGB2 gene, has been identified in the multi-site model but not recognized as significant at FDR=0.05 in the single-site analysis. The association between ITGB2 and kidney function has been supported by various data such as blood DNA methylation, RNA expression and expression quantitative trait loci (eQTLs) inhuman kidney samples, and single-cell RNA expression in mouse kidneys. The ITGB2 gene encodes integrin subunit beta 2 (also known as archetypal innate immune receptor CD11b/CD18), which plays an important role in immune response, and defects in this gene cause leukocyte adhesion deficiency. A recent study reported that inhibition of CD11b/CD18 prevented long-term fibrotic kidney failure from acute kidney injury (AKI) in cynomolgus monkeys.

Interestingly, our analysis identified several novel CpG sites associated with baseline eGFR with nearby genes having differential expression between samples from people with and without kidney disease. For example, both our single-site and multi-site analyses identified cg00506299 as being associated with baseline eGFR. This site is located within the RFTN1 gene, the methylation level of which has not been reported to be associated with kidney function previously. However, RFTN1 was found differentially expressed between DKD and controls and correlated with cortical interstitial fractional volume (Vvlnt) in DKD patients. In folic acid nephropathy (FAN) mouse kidneys, Rftn1 is also differentially expressed as compared to kidneys from healthy mice. As another example, cg21919729, located within the CTSB gene and identified by our single-site analysis, did not have its methylation reported to be associated with kidney disease previously, but its expression was found correlated with VvInt in DKD patients, and its mouse homologous gene Ctsb was differentially expressed in proximal tubule (PT) cells between FAN mice and healthy controls. CTSB encodes cathepsin B, a member of the C1 family of peptidases, which produces a lysosomal cysteine protease with both endopeptidase and exopeptidase activity that may play a role in protein turnover. Cathepsin B was reported to be involved in inflammation, apoptosis and autophagy during ESKD, CKD and AKI.

For eGFR slope, 52 of the 76 CpG sites (68.4%) were reported as potentially related to kidney function in the previous studies (FIG. 15), which corresponds to a 1.75-fold enrichment as compared to the set of all human genes (p=2.36×10⁻⁷, hypergeometric test).

One CpG site, cg19693031, which was selected by our multi-site model but not recognized as significant at FDR=0.05 in the single-site analysis, is located in the 3′-UTR (untranslated region) of the TXNIP gene. TXNIP encodes thioredoxin-interacting protein, which has been shown to play an important role in the pathogenesis of diabetic kidney disease. CpG sites within this gene were differentially methylated between baseline and 16-17 years follow-up between T1D patients with and without complications. TXNIP expression was also reported to be related to DKD, VvInt and FAN. Previous studies have found that hyperglycemia was able to up-regulate the level of inflammatory factors by up-regulating the expression of TXNIP through histone modifications such as increase in H3K9ac, H3K4me3, and H3K4me1, and decrease in H3K27me3 at TXNIP promoter region, consequently contributing to diabetic nephropathy. How DNA methylation is involved in this process requires further investigations. Another CpG site, cg13591783, identified in both our single-site and multi-site analyses for eGFR slope, is located within the ANXA1 gene. ANXA1 encodes annexin A1, which is a membrane-localized protein that binds phospholipids, inhibits phospholipase A2, and has anti-inflammatory activity. ANXA1 was found differentially expressed in kidney tubules between DKD and control samples and correlated with VvInt in DKD patients. Additionally, annexin A1 was a potential therapeutic target in diabetes and the treatment of microvascular disease such as diabetic nephropathy.

Taken together, among the genes near the CpG sites we found to be associated with baseline eGFR or eGFR slope in our single-site and multi-site analyses, many of them were previously reported to be related to normal kidney function or kidney diseases. These results were obtained based on by various types of data, including data produced from kidney samples, which provides strong support for the functional relevance of our reported CpG sites obtained from blood samples.

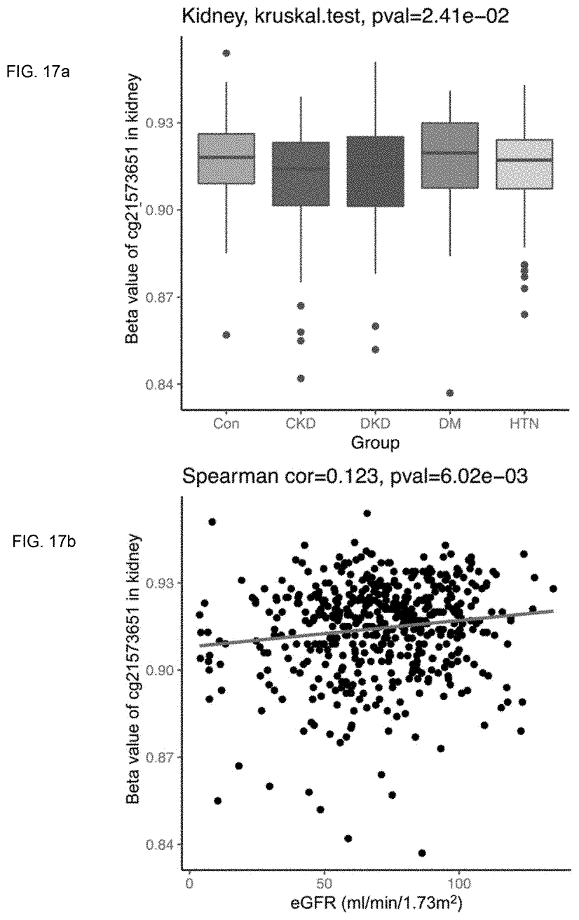

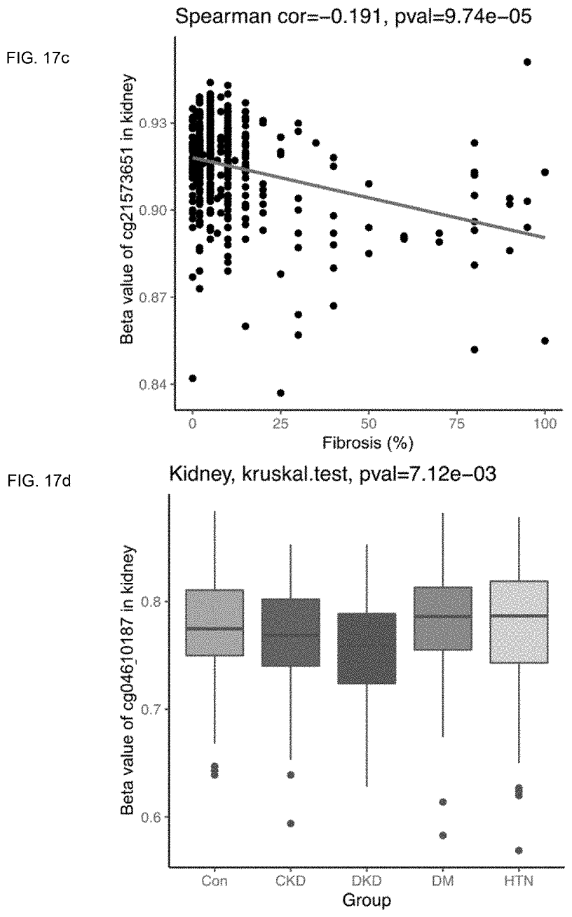

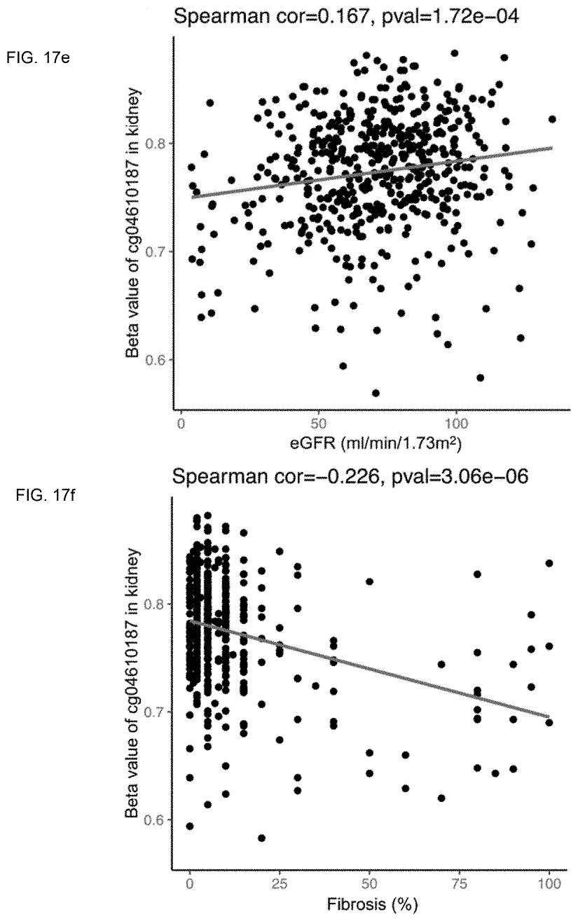

To further validate the relevance of our selected CpG sites in kidney, we selected seven CpG sites that were associated with baseline eGFR in our single-site and multi-site analyses, namely cg21573651, cg17944885, cg06449934, cg02304370, cg21919729, cg04610187 and cg18593194. For two of these seven CpG sites (cg21573651 and cg04610187) their methylation levels in kidney samples were significantly different between kidney disease patients and control groups (FIGS. 17a, 17d). Their methylation levels in kidney samples also had significant correlations with eGFR and fibrosis (FIGS. 17b-17c, 17e-17f). These results further supported that the CpG sites we identified from blood samples had functional significance in the kidney. In a different cohort of 84 individuals with type 2 diabetes from the Pima Indian population, two out of the 7 CpG sites identified (cg02304370 and cg18593194) showed suggestive association between methylation measured in peripheral blood with global glomerular sclerosis on morphometric variables of kidney biopsy samples in the same individuals (Table 10), again highlighting potential link between methylation level in blood and kidney pathology.

In an independent nested case-control cohort of 181 Pima Indians with type 2 diabetes, of which 80 developed ESRD during follow-up, baseline methylation scores for baseline eGFR or eGFR slope were both associated with incident ESRD (Table 11). The association was rendered non-significant after inclusion of baseline eGFR into the model, highlighting that the ability of the methylation changes to predict incident ESRD was mediated by methylation changes associated with baseline eGFR.

### DISCUSSION

In this study of methylation profiles from a cohort of patients with type 2 diabetes, our major findings are as follows: 1) DNA methylation level was associated with renal function in type 2 diabetes; 2) we were able to identify novel CpG sites for which methylation levels were associated with baseline eGFR; 3) we also identified a different set of 8 novel CpG sites which are associated with the rate of eGFR decline; 4) using methylation data, we were able to construct prediction models for baseline eGFR and decline in eGFR which were replicated in independent cohorts with type 2 diabetes; and 5) several of the key genes identified was found to be related to pathways important in the pathogenesis of kidney diseases.

Our results extend earlier work by others in highlighting the potential link between renal function and methylation profile. In particular, when compared against published studies of epigenome-wide association study for renal function, there was a degree of consistency whereby the top site identified in our study, cg17944885, near ZNF20, corresponds to a CpG site identified in several other EWAS for renal function. Furthermore, several other CpG sites identified in other studies to have their methylation levels associated with renal function in the general population were also found to show nominal association in our analysis of methylation changes. Interestingly, the replication of these findings from studies in the general population suggest that methylation changes associated with renal function in the general population may also be applicable to a population with type 2 diabetes. Furthermore, the earlier EWAS studies are predominantly from European populations, highlighting the advantage of methylation profiles whereby findings may not be ethnic-specific, as in the case of genetic loci identified from GWAS. Several of our findings identified in the current study were also identified in a recent meta-analysis of EWAS, but not identified in the earlier individual cohort studies. This may reflect improved statistical power from the recent larger meta-analysis, though it would warrant further investigation regarding whether transethnic meta-analysis is amore powerful strategy for discovering sites that are relevant across different ethnic populations.

In general, there was greater consistency for findings relating to methylation changes associated with baseline eGFR compared to decline in renal function. This is not surprising, given that key renal and other vascular pathology is likely to have a direct effect on modulating kidney function, though the rate of decline in kidney function would be more variable, and also subjected to various clinical factors including drug treatment, as well as the control of key risk factors such as blood pressure, lipids and glycaemia. Nevertheless, whilst it is difficult in a cross-sectional study to disentangle the relationship between methylation changes and renal function, and whether the methylation changes are simply consequences of the altered metabolic milieu related to renal dysfunction. On the other hand, methylation changes predictive of renal function decline, which seem to show minimal overlap with sites associated with baseline eGFR, are more likely to be of use as prognostic biomarkers.

Although we identified a number of methylation sites strongly associated with renal function and decline in renal function which reached stringent threshold of statistical significance after considering the number of statistical tests, the construction of a prediction model did not necessarily include all of these individually-significant CpG sites. This may appear surprising at first. Nevertheless, individual CpG sites may be strongly correlated with each other, due to spatial dependency or other reasons, leading to redundancy, as highlighted earlier.

The prediction model with the best performance generated using our data involved a combination of multiple CpG sites, many of which were not individually strongly associated with eGFR or eGFR decline. This approach of prediction models incorporating multiple sites versus ones that only include top individual CpG sites is somewhat analogous to the recent development of genome-wide polygenic risk scores, which tend to have better performance and utility, compared to the traditional approach of developing polygenic risk scores based on only GWAS-significant hits. Given the large number of methylation data sets currently available, our approach may be applicable for developing other prediction models based on epigenome-wide methylation data, an approach taken by the pioneering work of epigenetic clocks.

Our data highlight the potential utility of using methylation levels in blood samples to predict eGFR or change in eGFR. Note that these models incorporating methylation data performed significantly better than models incorporating only clinical variables. Previous studies of adding genetic variables, or other biomarkers, to clinical variables for prediction of diabetes-related complications have in general noted minimal improvement in prediction, suggesting that this approach in incorporating methylation data may be more fruitful in the long-run, and may capture disease risk that is beyond that captured by clinical risk factors themselves.

Tables

TABLE 1
Criteria for defining binary classes for clinical variables.
BMI: body mass index; FBG: fasting blood glucose; CS: current
smokers; NS: non-smokers; ES: ex-smoker; LDL: LDL-cholesterol;
HDL: HDL-cholesterol; TG: triglycerides; ACR: albumin-creatinine-
ratio; BP: blood pressure; SBP: systolic blood pressure;
DBP: diastolic blood pressure; HB: haemoglobin; LLD: lower-
lipid drugs. RASi: ACEI/ARB drugs.

| Clinical variable | Class 0 | Class 1 |
|---|---|---|
| Sex | Male | Female |
| Age (years) | <40 | ≥40 |
| Duration of diabetes (years) | <10 | ≥10 |
| BMI (kg/m²) | <25 | ≥25 |
| HbA1c (%) | <7 | ≥7 |
| FBG (mmol/L) | <7 | ≥7 |
| Smoking | CS | NS or ES |
| LDL (mmol/L) | <2.6 | ≥2.6 |
| HDL (mmol/L) | | |
| Female: | <1.3 | ≥1.3 |
| Male: | <1.0 | ≥1.0 |
| TG (mmol/L) | <1.7 | ≥1.7 |
| eGFR (ml/min/1.73 m²) | <60 | ≥60 |
| ACR | <30 | ≥30 |
| BP (mm Hg) | SBP < 130 and | SBP ≥ 130 or |
| | DBP < 80 | DBP ≥ 80 |
| HB (g/dL) | | |
| Female: | <11 | ≥11 |
| Male: | <13 | ≥13 |
| Use of LLD | Yes | No |
| Use of RASi | Yes | No |
| Use of insulin | Yes | No |
| Use of anti-hypertensive drugs | Yes | No |

TABLE 2
Mean AUROCs of different models using top 50 PCs for
classifying clinical variables. LR: logistic regression;
SVM: support vector machine; RF: random forest.

| | Mean AUROC | | |
|---|---|---|---|
| Clinical variables | LR | SVM | RF |
| Sex | 0.99 | 0.98 | 0.99 |
| Age | 0.95 | 0.82 | 0.86 |
| Duration of diabetes | 0.52 | 0.54 | 0.52 |
| BMI | 0.48 | 0.48 | 0.49 |
| HbA_{1c} | 0.57 | 0.55 | 0.57 |
| FBG | 0.45 | 0.51 | 0.50 |
| Smoking | 0.82 | 0.69 | 0.73 |
| LDL | 0.57 | 0.53 | 0.52 |
| HDL | 0.60 | 0.57 | 0.59 |
| TG | 0.54 | 0.52 | 0.50 |
| eGFR | 0.76 | 0.71 | 0.71 |
| ACR | 0.64 | 0.54 | 0.61 |
| BP | 0.59 | 0.55 | 0.56 |
| HB | 0.66 | 0.52 | 0.63 |
| Use of LLD | 0.54 | 0.49 | 0.49 |
| Use of RASi | 0.46 | 0.44 | 0.43 |
| Use of insulin | 0.56 | 0.52 | 0.52 |
| Use of anti-hypertensive drugs | 0.55 | 0.55 | 0.52 |

TABLE 3
Clinical characteristics of the participants
in the primary cohort. Data are shown as either a
single value and the corresponding percentage of
individuals with measurements, mean value standard
deviation, or median and the corresponding inter-
quartile range between the first and third quartiles.
Some variables (e.g., smoking status) contained some
missing values.
Number of samples before filtering 1,271
Number of samples after filtering 1,268

| Baseline characteristics | | |
|---|---|---|
| Male % (N) | 50.6% | (642) |
| Age (years) | 57.1 ± 11.3 | |
| Age of diabetes onset (years) | 49.2 ± 11.5 | |
| Duration of diabetes (years) | 7.9 ± 6.9 | |
| Smoking status % (N) | | |
| Non-smoker | 69.4% | (878) |
| Ex-smoker | 16.7% | (212) |
| Current smoker | 13.9% | (176) |
| Body height (m) | 1.59 ± 0.08 | |
| Body weight (kg) | 63.5 ± 11.9 | |
| Body mass index (kg/m²) | 25.1 ± 3.9 | |
| Waist circumference (cm) | | |
| Male | 87.7 ± 9.1 | |
| Female | 84.0 ± 9.8 | |
| Hip circumference (cm) | 96.3 ± 7.9 | |
| Waist-hip-ratio | 0.9 ± 0.1 | |
| HbA1c (%) | 7.9 ± 1.9 | |
| Total cholesterol (mmol/L) | 5.4 ± 1.3 | |
| Triglycerides (mmol/L) | 1.4 | (1.0-2.2) |
| HDL-cholesterol (mmol/L) | 1.3 ± 0.4 | |
| LDL-cholesterol (mmol/L) | 3.3 ± 1.11 | |
| Systolic blood pressure (mm Hg) | 137 ± 20.5 | |
| Diastolic blood pressure (mm Hg) | 77.3 ± 11.1 | |
| Hypertension % (N) | 74.2% | (941) |
| Retinopathy % (N) | 31.2% | (396) |
| Neuropathy % (N) | 23.1% | (293) |
| Microalbuminuria % (N) | 23.1% | (283) |
| Macroalbuminuria % (N) | 21.8% | (268) |
| Albumin-creatinine-ratio | 2.3 | (0.8-17.4) |
| eGFR (ml/min/1.73 m²) - CKD-EPI | 80.6 ± 25.0 | |
| Treatment | | |
| Lipid lowering drug % (N) | 13.8% | (175) |
| Blood pressure anti-hypertensive drug % (N) | 41.7% | (529) |
| ACE inhibitor/ARB % (N) | 20.0% | (253) |
| Oral glucose lowering drug % (N) | 61.5% | (780) |

TABLE 4
CpG sites with their methylation levels significantly associated with baseline eGFR or eGFR slope in the single-site
analysis. Each listed site has a Bonferroni-corrected p-value < 0.05. TSS1500: the region between 200 bp and 1,500 bp
upstream of the transcription start site (TSS). In the model coefficients, a positive sign means that a higher methylation
level is associated with higher baseline eGFR or slower eGFR decline, while a negative sign means the opposite.

| | | | | | | |
|---|---|---|---|---|---|---|
| CpG site | Genomic location | Model coefficient | P-value | Corrected p-value | Annotated gene(s) | Gene region(s) |

Baseline eGFR

| | | | | | | |
|---|---|---|---|---|---|---|
| cg17944885 | Chr19: 12,225,735 | −5.156 | 1.41E−20 | 6.11E−15 | — | — |
| cg25364972 | Chr2: 217,075,573 | −6.303 | 4.36E−11 | 1.90E−05 | — | — |
| cg06449934 | Chr7: 1,130,697 | 3.679 | 9.70E−11 | 4.22E−05 | GPER | 5′ UTR |
| | | | | | C7orf50 | Gene body |
| cg02304370 | Chr11: 587,926 | 3.662 | 1.37E−10 | 5.97E−05 | PHRF1 | Gene body |
| cg21919729 | Chr8: 11,719,367 | 3.368 | 4.28E−10 | 1.86E−04 | CTSB | 5′ UTR |
| cg04610187 | Chr17: 76,360,794 | 3.766 | 5.83E−10 | 2.53E−04 | — | — |
| cg04983687 | Chr16: 88,558,223 | 3.372 | 1.29E−09 | 5.61E−04 | ZFPM1 | Gene body |
| cg27254661 | Chr2: 73,118,624 | 3.697 | 2.47E−09 | 0.001 | SPR | Gene body |
| cg18593194 | Chr19: 36,205,201 | 3.697 | 2.75E−09 | 0.001 | ZBTB32 | 5′ UTR |
| cg12065228 | Chr1: 19,652,788 | 3.721 | 2.76E−09 | 0.001 | PQLC2 | Gene body |
| cg08940169 | Chr16: 88,540,241 | 3.260 | 4.16E−09 | 0.002 | ZFPM1 | Gene body |
| cg19434937 | Chr12: 7,104,184 | 3.206 | 4.16E−09 | 0.002 | LPCAT3 | Gene body |
| cg11699125 | Chr1: 6,341,327 | 3.144 | 6.55E−09 | 0.003 | ACOT7 | Gene body |
| cg17988187 | Chr2: 74,612,222 | 3.131 | 6.84E−09 | 0.003 | LOC100189589 | TSS1500 |
| cg09823543 | Chr6: 43,146,056 | 3.557 | 7.10E−09 | 0.003 | SRF | Gene body |
| cg02475695 | Chr16: 616,220 | 3.378 | 7.63E−09 | 0.003 | NHLRC4 | TSS1500 |
| cg06972908 | Chr16: 30,488,321 | 4.344 | 8.35E−09 | 0.004 | ITGAL | Gene body |
| cg11544657 | Chr1: 9,968,130 | −4.430 | 8.61E−09 | 0.004 | CTNNBIP1 | 5′ UTR |
| cg23845009 | Chr11: 34,323,678 | 4.360 | 1.09E−08 | 0.005 | ABTB2 | Gene body |
| cg09610644 | Chr3: 197,249,274 | −3.469 | 1.26E−08 | 0.005 | BDH1 | Gene body |
| cg12981272 | Chr3: 37,281,848 | 5.063 | 1.36E−08 | 0.006 | — | — |
| cg12077754 | Chr2: 75,089,669 | 3.114 | 1.38E−08 | 0.006 | HK2 | Gene body |
| cg10142874 | Chr2: 11,917,623 | 3.074 | 1.86E−08 | 0.008 | LPIN1 | Gene body |
| cg00934987 | Chr17: 56,605,468 | 3.540 | 2.68E−08 | 0.012 | SEPT4 | Gene body |
| cg22753611 | Chr6: 17,472,892 | −3.284 | 2.68E−08 | 0.012 | CAP2 | Gene body |
| cg04816311 | Chr7: 1,066,650 | 4.226 | 2.88E−08 | 0.013 | C7orf50 | Gene body |
| cg04497992 | Chr16: 616,212 | 3.053 | 3.11E−08 | 0.014 | NHLRC4 | TSS1500 |
| cg09249800 | Chr1: 6,341,287 | 3.042 | 3.15E−08 | 0.014 | ACOT7 | Gene body |
| cg01676795 | Chr7: 75,586,348 | 4.178 | 3.43E−08 | 0.015 | POR | Gene body |
| cg25854298 | Chr10: 73,936,754 | 2.952 | 3.79E−08 | 0.016 | ASCCI | Gene body |
| cg10489463 | Chr2: 33,546,572 | 3.190 | 4.07E−08 | 0.018 | LTBP1 | Gene body |
| cg23516680 | Chr10: 103,923,333 | 3.105 | 4.89E−08 | 0.021 | NOLC1 | 3′ UTR |
| cg02170785 | Chr14: 69,650,830 | 3.012 | 5.44E−08 | 0.024 | — | — |
| cg19448292 | Chr20: 35,504,064 | 3.177 | 5.59E−08 | 0.024 | C20orf118 | TSS1500 |
| cg01499988 | Chr9: 35,755,346 | 2.980 | 6.16E−08 | 0.027 | MSMP | TSS1500 |
| cg25087851 | Chr11: 60,623,918 | 2.993 | 6.95E−08 | 0.030 | GPR44 | TSS1500 |
| cg22406869 | Chr11: 66,276,941 | 4.239 | 7.63E−08 | 0.033 | DPP3 | 3′ UTR |
| | | | | | BBS1 | TSS1500 |
| cg18650626 | Chr7: 1,914,073 | 2.886 | 8.89E−08 | 0.039 | MAD1L1 | Gene body |
| cg00506299 | Chr3: 16,469,127 | 3.373 | 9.14E−08 | 0.040 | RFTN1 | Gene body |
| cg16809457 | Chr6: 90,399,677 | 3.694 | 1.14E−07 | 0.050 | MDN1 | Gene body |

eGFR slope

| | | | | | | |
|---|---|---|---|---|---|---|
| cg10272901 | Chr21: 46,677,879 | 1.316 | 7.84E−11 | 3.41E−05 | — | — |
| cg12354056 | Chr3: 186,136,503 | 1.126 | 7.50E−10 | 3.26E−04 | — | — |
| cg18461548 | Chr8: 37,701,921 | 1.179 | 2.72E−09 | 0.001 | BRF2 | 3′ UTR |
| cg00695821 | Chr3: 156,124,891 | 1.354 | 3.81E−09 | 0.002 | KCNAB1 | Gene body |
| cg22822893 | Chr6: 15,1662,789 | 1.056 | 7.39E−09 | 0.003 | AKAP12 | Gene body |
| cg02566611 | Chr16: 83,948,975 | 0.986 | 5.61E−08 | 0.024 | MLYCD | Gene body |
| cg20741134 | Chr1: 181,382,639 | 0.976 | 5.67E−08 | 0.025 | — | — |
| cg04027328 | Chr1: 11,372,138 | 1.290 | 6.81E−08 | 0.030 | — | — |
| cg25364972 | Chr2: 217,075,573 | −6.303 | 4.36E−11 | 1.90E−05 | — | — |

TABLE 5
CpG sites in the final multi-site model for baseline eGFR. Sites with a zero coefficient in a model are those that were
originally selected by our procedure as input for the LASSO method to consider but were finally not given a non-zero
weight. TSS200: the region between the transcription start site (TSS) and 200 bp upstream of it. TSS1500: the region
between 200 bp and 1,500 bp upstream of the TSS. In the model coefficients, a positive sign means that a higher
methylation level is associated with higher baseline eGFR or slower eGFR decline, while a negative

| | | Model coefficient | | | | |
|---|---|---|---|---|---|---|
| | | | Without | Single-site | | |
| CpG site | Genomic location | With covariates | covariates | corrected p-value | Annotated gene(s) | Gene region(s) |
| cg17944885 | Chr19: 12225735 | −3.291 | −4.211 | 6.11E−15 | — | — |
| cg06449934 | Chr7: 1130697 | 0.442 | 0.088 | 4.22E−05 | GPER | 5′ UTR |
| | | | | | C7orf50 | Gene body |
| cg02304370 | Chr11: 587926 | 0.491 | 0.313 | 5.97E−05 | PHRF1 | Gene body |
| cg21919729 | Chr8: 11719367 | 0.778 | 0.715 | 1.86E−04 | CTSB | 5′ UTR |
| cg04610187 | Chr17: 76360794 | 0.656 | 0.721 | 2.54E−04 | — | — |
| cg18593194 | Chr19: 36205201 | 1.661 | 1.188 | 0.001 | ZBTB32 | 5′ UTR |
| cg12065228 | Chr1: 19652788 | 0 | 0 | 0.001 | PQLC2 | Gene body |
| cg09823543 | Chr6: 43146056 | 1.127 | 1.047 | 0.003 | SRF | Gene body |
| cg23845009 | Chr11: 34323678 | 2.249 | 1.145 | 0.005 | ABTB2 | Gene body |
| cg09610644 | Chr3: 197249274 | −1.780 | −2.809 | 0.005 | BDH1 | Gene body |
| cg00934987 | Chr17: 56605468 | 0 | 0.661 | 0.012 | SEPT4 | Gene body |
| cg04497992 | Chr16: 616212 | 0.116 | 0 | 0.014 | NHLRC4 | TSS1500 |
| cg01676795 | Chr7: 75586348 | 1.939 | 1.225 | 0.015 | POR | Gene body |
| cg00506299 | Chr3: 16469127 | 1.464 | 0.713 | 0.040 | RFTN1 | Gene body |
| cg01885635 | Chr3: 40566085 | 1.877 | 3.159 | 0.169 | ZNF621 | TSS1500 |
| cg15232319 | Chr19: 4376459 | 0 | −0.557 | 0.414 | SH3GL1 | Gene body |
| cg20062057 | Chr2: 50201479 | 1.508 | 1.428 | 0.466 | NRXN1 | Gene body |
| cg07397612 | Chr22: 47423986 | 1.452 | 1.613 | 0.497 | TBCID22A | Gene body |
| cg20970369 | Chr1: 111744108 | −1.123 | −1.395 | 0.658 | DENND2D | TSS1500 |
| cg13091627 | Chr1: 153518476 | −1.825 | −1.504 | 0.851 | S100A4 | TSS200 |
| cg23511909 | Chr3: 128340787 | 0.555 | 0.722 | 0.887 | RPN1 | Gene body |
| cg02835823 | Chr16: 85979060 | −0.451 | 0 | 0.902 | — | — |
| cg20133890 | Chr6: 31680144 | 0 | 0 | 1 | LY6G6E | Gene body |
| cg12465678 | Chr1: 27953336 | 0.045 | −1.188 | 1 | FGR | TSS1500 |
| cg20299697 | Chr3: 138069423 | 0.764 | 1.401 | 1 | MRAS | 5′ UTR |
| cg14141741 | Chr7: 947428 | 1.157 | 0.893 | 1 | ADAP1 | Gene body |
| cg19458497 | Chr11: 63403371 | 0.848 | 0.972 | 1 | ATL3 | Gene body |
| cg10578938 | Chr5: 156695410 | −0.565 | −0.667 | 1 | CYFIP2 | 5′ UTR |
| cg22049753 | Chr2: 240895815 | 1.292 | 1.216 | 1 | — | — |
| cg26344619 | Chr14: 76046018 | 1.082 | 0.987 | 1 | FLVCR2 | Gene body |
| cg11845111 | Chr2: 191398756 | −1.155 | −1.506 | 1 | TMEM194B | Gene body |
| cg23509869 | Chr6: 31553441 | −1.424 | −0.488 | 1 | LST1 | TSS1500 |
| cg14583999 | Chr3: 10019040 | 0.691 | 1.162 | 1 | TMEM111 | Gene body |
| cg06943835 | Chr11: 64662577 | 0.734 | 1.908 | 1 | ATG2A | Gene body |
| cg19597449 | Chr19: 8117924 | 0.909 | 0 | 1 | CCL25 | TSS200 |
| cg26336935 | Chr17: 39769213 | 1.045 | 1.218 | 1 | KRT16 | TSS200 |
| cg23261820 | Chr5: 102382738 | 1.311 | 1.636 | 1 | — | — |
| cg07781445 | Chr17: 2886250 | 0 | 0.727 | 1 | RAPIGAP2 | Gene body |
| cg18036734 | Chr5: 177036766 | 0.495 | 0 | 1 | B4GALT7 | 3′ UTR |
| cg01924561 | Chr1: 43416103 | −1.267 | −1.538 | 1 | SLC2A1 | Gene body |
| cg07477034 | Chr17: 53341969 | 1.128 | 1.754 | 1 | HLF | TSS1500 |
| cg24707889 | Chr21: 46341304 | −0.252 | 0.217 | 1 | ITGB2 | 5′UTR |
| cg00501876 | Chr3: 39193251 | −2.161 | −1.533 | 1 | CSRNP1 | 5′UTR |
| cg25013303 | Chr1: 10961257 | 0.042 | 0.387 | 1 | — | — |
| cg18070458 | Chr11: 121319927 | −0.802 | −0.611 | 1 | — | — |
| cg11961845 | Chr7: 129008179 | −0.606 | −0.081 | 1 | AHCYL2 | Gene body |
| cg17124293 | Chr10: 45403981 | −1.490 | −1.360 | 1 | — | — |
| cg13408344 | Chr15: 31631240 | −0.665 | −0.627 | 1 | KLF13 | Gene body |
| cg19893929 | Chr2: 16105823 | −0.103 | 0 | 1 | — | — |
| cg00791074 | Chr6: 151186169 | 0 | 0.079 | 1 | MTHFD1L | TSS1500 |
| cg26608718 | Chr19: 15530737 | 0.238 | 1.443 | 1 | AKAP8L | TSS1500 |
| cg01955153 | Chr16: 50769852 | −0.380 | 0 | 1 | — | — |
| cg06015525 | Chr12: 57872123 | −1.678 | −1.772 | 1 | ARHGAP9 | Gene body |
| cg16324121 | Chr3: 9954273 | 0 | −1.235 | 1 | IL17RE | Gene body |
| cg05062653 | Chr5: 562341 | −1.604 | −1.597 | 1 | — | — |
| cg03881294 | Chr2: 11884333 | 0 | 0 | 1 | — | — |
| cg12171761 | Chr8: 61910949 | −0.200 | −0.349 | 1 | — | — |
| cg00912580 | Chr2: 135169533 | −0.107 | −0.145 | 1 | MGAT5 | Gene body |
| cg26687842 | Chr13: 41055491 | −1.335 | −1.991 | 1 | LOC646982 | TSS1500 |
| cg27376617 | Chr7: 30518048 | 1.132 | 1.501 | 1 | NOD1 | 5′ UTR |
| cg03032497 | Chr14: 61108227 | 0 | −1.895 | 1 | — | — |
| cg09511896 | Chr1: 228246937 | −1.370 | −1.690 | 1 | WNT3A | Gene body |
| cg03607117 | Chr3: 53080440 | −1.360 | −3.570 | 1 | SFMBT1 | TSS1500 |
| cg18473521 | Chr12: 54448265 | −0.651 | −1.655 | 1 | HOXC4 | Gene body |

TABLE 6
CpG sites in the final multi-site model for eGFR slope. Sites with a zero coefficient in a model are those that
were originally selected by our procedure as input for the LASSO method to consider but were finally not
given a non-zero weight. TSS200: the region between the transcription start site (TSS) and 200 bp upstream
of it. TSS1500: the region between 200 bp and 1,500 bp upstream of the TSS. In the model coefficients, a
positive sign means that a higher methylation level is associated with higher baseline eGFR or slower eGFR
decline, while a negative sign means the opposite.

| | | Model coefficient | | | | |
|---|---|---|---|---|---|---|
| | | With | Without | Single-site | | |
| CpG site | Genomic location | covariates | covariates | corrected p-value | Annotated gene(s) | Gene region(s) |
| cg10272901 | Chr21: 46677879 | 0.684 | 0.679 | 3.41E−05 | — | — |
| cg12354056 | Chr3: 186136503 | 0.255 | 0.345 | 3.26E−04 | — | — |
| cg22822893 | Chr6: 151662789 | 0.075 | 0.035 | 0.003 | AKAP12 | Gene body |
| cg04027328 | Chr1: 11372138 | 0.243 | 0.005 | 0.030 | — | — |
| cg16425726 | Chr4: 83680145 | 0.403 | 0.385 | 0.050 | SCD5 | Gene body |
| cg21368479 | Chr6: 149415018 | 0.702 | 0.683 | 0.055 | — | — |
| cg22930808 | Chr3: 122281881 | 0.386 | 0.352 | 0.063 | PARP9 | 5′ UTR |
| | | | | | DTX3L | TSS1500 |
| cg01647632 | Chr15: 89438905 | 0.477 | 0.476 | 0.350 | HAPLN3 | TSS200 |
| cg13591783 | Chr9: 75768868 | 0.598 | 0.625 | 0.429 | ANXA1 | 5′ UTR |
| cg10761425 | Chr3: 12988976 | −0.575 | −0.517 | 0.991 | IQSEC1 | Gene body |
| cg15989436 | Chr5: 150465875 | 0.110 | 0 | 1 | — | — |
| cg23047271 | Chr3: 64210991 | 0.476 | 0.615 | 1 | PRICKLE2 | First exon |
| cg02647990 | Chr3: 196230837 | 0.612 | 0.553 | 1 | RNF168 | TSS1500 |
| cg05580141 | Chr12: 49071788 | 0 | −0.153 | 1 | C12orf41 | Gene body |
| cg17944885 | Chr19: 12225735 | −0.758 | −1.061 | 1 | — | — |
| cg04383715 | Chr16: 34209247 | 0.662 | 0.653 | 1 | — | — |
| cg14943908 | Chr6: 31589196 | 0 | −0.049 | 1 | BAT2 | 5′ UTR |
| cg07723558 | Chr17: 7184224 | 0.383 | 0.456 | 1 | SLC2A4 | TSS1500 |
| cg06575692 | Chr16: 68112968 | −0.494 | −0.615 | 1 | DUS2L | 3′ UTR |
| cg11494773 | Chr7: 48128242 | 0 | 0.197 | 1 | UPP1 | TSS200 |
| cg16933224 | Chr11: 63604740 | 0.141 | 0.336 | 1 | — | — |
| cg25686812 | Chr3: 42597657 | −0.286 | −0.298 | 1 | SEC22C | Gene body |
| cg04697209 | Chr16: 20087376 | −0.538 | −0.627 | 1 | — | — |
| cg12526474 | Chr7: 140097579 | 0.147 | 0.314 | 1 | SLC37A3 | 5′ UTR |
| cg06681597 | Chr17: 13972703 | −0.611 | −0.725 | 1 | COX10 | TSS200 |
| cg20010135 | Chr16: 30996822 | 0 | 0.084 | 1 | HSD3B7 | 5′ UTR |
| cg20101066 | Chr7: 148581385 | −0.607 | −0.690 | 1 | EZH2 | 5′ UTR |
| cg08626625 | Chr6: 33129765 | 0.107 | −0.034 | 1 | — | — |
| cg21926091 | Chr8: 141108607 | −0.031 | −0.300 | 1 | TRAPPC9 | Gene body |
| cg15581429 | Chr19: 39369353 | −0.648 | −0.458 | 1 | SIRT2 | 3′ UTR |
| cg19693031 | Chr1: 145441552 | 0.931 | 1.428 | 1 | TXNIP | 3′ UTR |
| cg21693780 | Chr2: 15731793 | 0 | 0.109 | 1 | DDX1 | First exon |
| cg10639435 | Chr8: 146104221 | −0.143 | −0.383 | 1 | ZNF250 | 3′ UTR |
| cg12245040 | Chr16: 2009320 | 0.019 | 0.145 | 1 | NDUFB10 | TSS200 |
| cg05166473 | Chr16: 88103629 | −0.371 | −0.293 | 1 | BANP | Gene body |
| cg20728490 | Chr10: 98064175 | −0.145 | −0.090 | 1 | DNTT | 5′ UTR |
| cg22293458 | Chr3: 184483865 | −0.550 | −0.493 | 1 | — | — |

TABLE 7
Performance of the multi-site models constructed from data of the primary cohort and applied
to either the primary or Pima Indian cohort. The “CpG sites” column shows the number of sites
selected by our procedure as input for the LASSO method to consider, some of which finally got
assigned a zero weight by LASSO.

| Testing cohort | Target phenotype | CpG sites | Covariates | PCC | SCC | MAE |
|---|---|---|---|---|---|---|
| Primary | Baseline eGFR | 64 | Yes | 0.806 | 0.762 | 11.707 |
| | | | No | 0.765 | 0.717 | 12.815 |
| | eGFR slope | 37 | Yes | 0.635 | 0.584 | 4.119 |
| | | | No | 0.589 | 0.532 | 4.327 |
| Primary (only CpG sites | Baseline eGFR | 59 | Yes | 0.801 | 0.759 | 11.838 |
| common to both cohorts) | | | No | 0.759 | 0.712 | 12.957 |
| | eGFR slope | 29 | Yes | 0.612 | 0.564 | 4.202 |
| | | | No | 0.562 | 0.507 | 4.430 |
| Pima Indians | Baseline eGFR | 59 | Yes | 0.591 | 0.614 | 26.947 |
| | | | No | 0.497 | 0.534 | 27.528 |
| | eGFR slope | 29 | Yes | 0.356 | 0.389 | 4.260 |
| | | | No | 0.273 | 0.279 | 4.274 |

PCC: Pearson correlation coefficient,
SCC: Spearman correlation coefficient,
MAE: mean absolute error.

TABLE 8
Performance of regression models using different sets of CpG sites
as input. The input CpG sites of the alternative models are defined
in the Results section. All results shown here were determined based
on 5-fold cross-validation. PCC: Pearson correlation coefficient;
SCC: Spearman correlation coefficient; MAE: mean absolute error

| Input CpG sites | Covariates | PCC | SCC | MAE |
|---|---|---|---|---|
| Baseline eGFR | | | | |
| All | Yes | 0.762 | 0.718 | 12.598 |
| | No | 0.719 | 0.672 | 13.644 |
| Corrected p < 0.05 | Yes | 0.699 | 0.674 | 13.986 |
| | No | 0.551 | 0.492 | 16.990 |
| Significant at FDR = 0.05 | Yes | 0.743 | 0.702 | 13.078 |
| | No | 0.662 | 0.593 | 14.955 |
| Most significant | Yes | 0.715 | 0.681 | 13.751 |
| | No | 0.600 | 0.533 | 16.141 |
| Covariates only | Yes | 0.621 | 0.624 | 14.973 |
| eGFR slope | | | | |
| All | Yes | 0.551 | 0.502 | 4.427 |
| | No | 0.528 | 0.470 | 4.541 |
| Corrected p < 0.05 | Yes | 0.399 | 0.380 | 4.822 |
| | No | 0.219 | 0.200 | 5.425 |
| Significant at FDR = 0.05 | Yes | 0.451 | 0.444 | 4.648 |
| | No | 0.343 | 0.321 | 5.080 |
| Most significant | Yes | 0.450 | 0.453 | 4.619 |
| | No | 0.339 | 0.343 | 5.054 |
| Covariates only | Yes | 0.368 | 0.369 | 4.871 |

TABLE 9
Performance of classification models using different sets of
CpG sites as input. The input CpG sites of the alternative
models are defined in the Results section. Binary class threshold
is 60 and −4 for baseline eGFR and eGFR slope, respectively.
All results shown here were determined based on 10-fold cross-
validation (stratified with class labels).

| Input CpG sites | Covariates | mean AUROC |
|---|---|---|
| Baseline eGFR | | |
| All | Yes | 0.893 |
| | No | 0.883 |
| Corrected p < 0.05 | Yes | 0.885 |
| | No | 0.825 |
| Significant at FDR = 0.05 | Yes | 0.897 |
| | No | 0.876 |
| Most significant | Yes | 0.875 |
| | No | 0.841 |
| Covariates only | Yes | 0.832 |
| eGFR slope | | |
| All | Yes | 0.805 |
| | No | 0.780 |
| Corrected p < 0.05 | Yes | 0.756 |
| | No | 0.627 |
| Significant at FDR = 0.05 | Yes | 0.782 |
| | No | 0.706 |
| Most significant | Yes | 0.772 |
| | No | 0.701 |
| Covariates only | Yes | 0.750 |

TABLE 10
Correlation between DNA methylation levels of our seven selected CpG sites in blood and morphometric
variables from kidney biopsies in the same individuals. For each variable, the first row (with prefix “r_” added
to the variable name) shows the partial Pearson correlations and the second row (with prefix “p_” added to the
variable name) shows the p-values. P-values smaller than or equal to 0.05 are in bold face.

| | cg21573651 | cg17944885 | cg06449934 | cg02304370 | cg21919729 | cg04610187 | cg18593194 |
|---|---|---|---|---|---|---|---|
| r_FPW | 0.04 | −0.19 | −0.05 | 0.01 | −0.08 | 0.12 | −0.23 |
| p_FPW | 0.74 | 0.12 | 0.70 | 0.95 | 0.50 | 0.34 | 0.07 |
| r_GBM | −0.08 | 0.01 | −0.09 | −0.06 | 0.05 | 0.10 | 0.04 |
| p_GBM | 0.52 | 0.96 | 0.45 | 0.62 | 0.68 | 0.44 | 0.74 |
| r_GS | 0.04 | −0.14 | −0.06 | −0.29 | 0.04 | −0.07 | −0.25 |
| p_GS | 0.76 | 0.25 | 0.63 | 0.01 | 0.75 | 0.55 | 0.03 |
| r_GV | 0.06 | −0.05 | 0.14 | −0.03 | 0.12 | 0.08 | 0.10 |
| p_GV | 0.64 | 0.68 | 0.23 | 0.77 | 0.30 | 0.49 | 0.38 |
| r_MEAN_N_E | 0.01 | −0.04 | 0.13 | −0.03 | 0.06 | 0.09 | 0.10 |
| p_MEAN_N_E | 0.92 | 0.75 | 0.27 | 0.82 | 0.62 | 0.47 | 0.39 |
| r_PCT_FENE | 0.08 | −0.01 | −0.17 | 0.01 | 0.14 | −0.06 | 0.14 |
| p_PCT_FENE | 0.51 | 0.95 | 0.15 | 0.92 | 0.24 | 0.60 | 0.25 |
| r_SV | −0.08 | 0.20 | 0.04 | 0.05 | 0.05 | 0.05 | 0.08 |
| p_SV | 0.49 | 0.10 | 0.76 | 0.69 | 0.67 | 0.68 | 0.50 |
| r_VVINT | 0.08 | 0.03 | −0.02 | −0.05 | −0.08 | 0.00 | 0.00 |
| p_VVINT | 0.52 | 0.78 | 0.88 | 0.66 | 0.51 | 0.98 | 1.00 |
| r_VVMES | −0.10 | 0.00 | 0.04 | 0.08 | 0.12 | 0.07 | 0.00 |
| p_VVMES | 0.38 | 0.97 | 0.72 | 0.50 | 0.34 | 0.59 | 0.99 |

FPW: podocyte foot process width (nm),
GBM: glomerular basement membrane width (nm),
GS: global glomerular sclerosis (%),
GV: mean glomerular volume (× 10⁶ μm³),
MEAN_N_E: non-podocyte number per glomerulus (N),
PCT_FENE: percent fenestrated endothelium (%),
SV: glomerular filtration surface density (μ²/μ³),
VVINT: cortical interstitial fractional volume (%),
VVMES: mesangial fractional volume (%).

TABLE 11
Associations of baseline methylation score with incident ESRD in American Indian
nested case-control study. Based on nested case-control study with 80 incident
ESRD cases and 181 total individuals. Methylation score for baseline eGFR is
based on 64 available CpG sites, while the score for eGFR slope is based on 37
available CpG sites. Hazard ratios (HR) are expressed per SD of the methylation.
Correlations with baseline eGFR are 0.69 and 0.64 for baseline eGFR target methylation
score with and without covariates respectively; corresponding correlations for
the eGFR slope methylation score are 0.22 and 0.26, respectively.

| | Base model | | Base model + baseline eGFR | |
|---|---|---|---|---|
| Target phenotype | HR (95% CI) | p-value | HR (95% CI) | p-value |
| Baseline eGFR, without covariates | 0.59 (0.41, 0.84) | 0.0037 | 1.01 (0.66, 1.54) | 0.9714 |
| Baseline eGFR, with covariates | 0.66 (0.49, 0.90) | 0.0078 | 1.04 (0.73, 1.49) | 0.8188 |
| eGFR slope, without covariates | 0.75 (0.58, 0.97) | 0.0307 | 0.90 (0.67, 1.20) | 0.4767 |
| eGFR slope, with covariates | 0.77 (0.60, 1.00) | 0.0518 | 0.94 (0.71, 1.26) | 0.6807 |

Supplementary Table 5: left table shows baseline eGFR without
covariate and right table shows baseline eGFR with covariate

| CpG site | Coefficient | CpG site | Coefficient |
|---|---|---|---|
| cg18593194 | 1.187981341 | cg18593194 | 1.661481056 |
| cg17944885 | −4.210748418 | cg17944885 | −3.291003261 |
| cg04610187 | 0.720838582 | cg04610187 | 0.656165623 |
| cg13091627 | −1.504232244 | cg13091627 | −1.825272138 |
| cg23845009 | 1.144588915 | cg02835823 | −0.451262666 |
| cg00912580 | −0.145003095 | cg23845009 | 2.248872096 |
| cg03607117 | −3.570230939 | cg00912580 | −0.106733458 |
| cg10578938 | −0.66684641 | cg03607117 | −1.359668407 |
| cg26608718 | 1.44257369 | cg10578938 | −0.565489697 |
| cg21919729 | 0.715355086 | cg26608718 | 0.238380525 |
| cg18070458 | −0.611108746 | cg21919729 | 0.778239465 |
| cg24707889 | 0.217438765 | cg19597449 | 0.908707717 |
| cg00506299 | 0.713228389 | cg18070458 | −0.801682972 |
| cg13408344 | −0.627229282 | cg24707889 | −0.252408915 |
| cg09610644 | −2.808517299 | cg00506299 | 1.464356932 |
| cg14583999 | 1.161955594 | cg13408344 | −0.665418868 |
| cg14141741 | 0.893314163 | cg09610644 | −1.780353113 |
| cg00791074 | 0.078815788 | cg14583999 | 0.690851449 |
| cg01676795 | 1.225165483 | cg14141741 | 1.15675953 |
| cg20970369 | −1.395116131 | cg01676795 | 1.939030439 |
| cg11961845 | −0.080765308 | cg18036734 | 0.495461944 |
| cg20299697 | 1.400604624 | cg20970369 | −1.123303117 |
| cg23509869 | −0.487645261 | cg11961845 | −0.605987309 |
| cg07397612 | 1.613085839 | cg20299697 | 0.764424062 |
| cg27376617 | 1.500864179 | cg23509869 | −1.424398348 |
| cg01885635 | 3.158944134 | cg07397612 | 1.451688001 |
| cg26336935 | 1.217978667 | cg27376617 | 1.13203033 |
| cg06943835 | 1.907978271 | cg01885635 | 1.876510006 |
| cg12171761 | −0.349230535 | cg26336935 | 1.045253451 |
| cg09823543 | 1.047142778 | cg06943835 | 0.734126043 |
| cg06449934 | 0.088173968 | cg12171761 | −0.200135012 |
| cg19458497 | 0.972434521 | cg09823543 | 1.126736677 |
| cg15232319 | −0.55722739 | cg06449934 | 0.442383987 |
| cg22049753 | 1.215882502 | cg19458497 | 0.84765765 |
| cg09511896 | −1.690177727 | cg01955153 | −0.38032517 |
| cg20062057 | 1.427853994 | cg22049753 | 1.292403435 |
| cg01924561 | −1.538274174 | cg09511896 | −1.370120713 |
| cg00934987 | 0.661461099 | cg20062057 | 1.50771785 |
| cg23511909 | 0.722246069 | cg01924561 | −1.266649123 |
| cg05062653 | −1.596827394 | cg04497992 | 0.116232467 |
| cg11845111 | −1.505917398 | cg23511909 | 0.554847566 |
| cg17124293 | −1.360253384 | cg05062653 | −1.604169028 |
| cg26687842 | −1.991065501 | cg11845111 | −1.154624651 |
| cg06015525 | −1.77194467 | cg17124293 | −1.489990035 |
| cg03032497 | −1.894683345 | cg26687842 | −1.335457878 |
| cg26344619 | 0.987025099 | cg06015525 | −1.678317465 |
| cg16324121 | −1.234809317 | cg26344619 | 1.081805849 |
| cg23261820 | 1.635725474 | cg23261820 | 1.311135301 |
| cg00501876 | −1.53303399 | cg00501876 | −2.160608718 |
| cg02304370 | 0.313039803 | cg02304370 | 0.491150574 |
| cg12465678 | −1.187503442 | cg19893929 | −0.102540389 |
| cg07781445 | 0.727037665 | cg12465678 | 0.044777105 |
| cg07477034 | 1.754136143 | cg07477034 | 1.128394063 |
| cg18473521 | −1.655292422 | cg18473521 | −0.651469892 |
| cg25013303 | 0.387299367 | cg25013303 | 0.042282398 |
| | | AGE | −5.588496862 |
| | | SMOKING_new | 0.119048706 |
| | | DMAGE | −2.1808697 |
| | | HBA1C | −0.571126149 |
| | | SBP | −3.432158914 |
| | | DBP | 0.748769895 |
| | | CD8T | −0.852180511 |
| | | CD4T | −1.798515698 |
| | | Mono | 0.573178182 |
| | | Gran | 2.877802215 |
| | | sentrix_pos | 0.625355406 |
| | | sample_plate | −0.106976461 |
| Intercept | 80.5936 | Intercept | 80.5936 |

Supplementary Table 6: left table shows eGFR slope without
covariate and right table shows eGFR slope with covariate

| CpG site | Coefficient | CpG site | Coefficient |
|---|---|---|---|
| cg10639435 | −0.382638274 | cg10639435 | −0.142610646 |
| cg13591783 | 0.624771678 | cg13591783 | 0.59833222 |
| cg10761425 | −0.517070477 | cg10761425 | −0.575039098 |
| cg12354056 | 0.345441868 | cg12354056 | 0.254999677 |
| cg11494773 | 0.197233511 | cg19693031 | 0.930587908 |
| cg19693031 | 1.428298862 | cg01647632 | 0.476794678 |
| cg01647632 | 0.475753109 | cg10272901 | 0.684262026 |
| cg10272901 | 0.678755235 | cg04027328 | 0.24281183 |
| cg04027328 | 0.005410375 | cg15989436 | 0.110076173 |
| cg06681597 | −0.725406789 | cg06681597 | −0.6114486 |
| cg22930808 | 0.351814679 | cg22930808 | 0.385955082 |
| cg20010135 | 0.08414898 | cg21368479 | 0.702270799 |
| cg21368479 | 0.683027114 | cg06575692 | −0.49395046 |
| cg06575692 | −0.615207691 | cg16425726 | 0.402654965 |
| cg16425726 | 0.384811469 | cg20728490 | −0.144523722 |
| cg20728490 | −0.090202283 | cg17944885 | −0.757667851 |
| cg17944885 | −1.060522203 | cg25686812 | −0.285989524 |
| cg25686812 | −0.298251333 | cg12526474 | 0.146951343 |
| cg12526474 | 0.313602502 | cg22293458 | −0.55000994 |
| cg14943908 | −0.048886796 | cg07723558 | 0.382952467 |
| cg22293458 | −0.493253816 | cg04383715 | 0.662225559 |
| cg05580141 | −0.152923984 | cg02647990 | 0.611964518 |
| cg07723558 | 0.455682147 | cg21926091 | −0.030698563 |
| cg04383715 | 0.652786402 | cg08626625 | 0.107363249 |
| cg02647990 | 0.553390828 | cg04697209 | −0.537886758 |
| cg21693780 | 0.108501537 | cg23047271 | 0.47581982 |
| cg21926091 | −0.300497177 | cg15581429 | −0.648195034 |
| cg08626625 | −0.033686738 | cg05166473 | −0.371202726 |
| cg04697209 | −0.627425327 | cg12245040 | 0.018812834 |
| cg23047271 | 0.614951461 | cg20101066 | −0.606783129 |
| cg15581429 | −0.457749392 | cg22822893 | 0.07517686 |
| cg05166473 | −0.29259304 | cg16933224 | 0.140957651 |
| cg12245040 | 0.145211315 | | |
| cg20101066 | −0.690050887 | | |
| cg22822893 | 0.035465479 | AGE | 0.244448442 |
| cg16933224 | 0.335625662 | SMOKING_new | −0.042569077 |
| | | DMAGE | −0.777896261 |
| | | SBP | −1.176248086 |
| | | DBP | 0.2200314 |
| | | CD8T | −0.25995336 |
| | | Bcell | −0.047390684 |
| | | Mono | 0.073969228 |
| | | Gran | 0.453934013 |
| | | sentrix_code | −0.427133542 |
| | | sample_well | −0.26742055 |
| Intercept | −5.69909 | Intercept | −5.74496 |

What is claimed is:

1. A method for determining a total methylation level of one or more CpG sites in a subject, comprising:
(a) extracting DNA from a biological sample obtained from the subject;
(b) performing an assay by contacting the DNA with reagents hybridizing to the one or more CpG sites, wherein the one or more CpG sites are selected from the group consisting of cg10272901, cg12354056, cg18461548, cg00695821, cg22822893, cg02566611, cg20741134, cg04027328, cg21573651, cg17944885, cg06449934, cg02304370, cg21919729, cg04610187 and cg18593194;
(c) detecting a total number of the one or more CpG sites based on the signals obtained from the assay; and
(d) determining the total methylation level of the one or more CpG sites using the total number.

2. The method of claim 1, wherein the subject has already had diabetes, such as type 1 diabetes (T1D) or type 2 diabetes (T2D).

3. The method of claim 1, wherein the reagents hybridizing to the one or more CpG sites are those involved in methods selected from the group consisting of High-performance Liquid Chromatography (HPLC), High-performance Capillary Electrophoresis (HPCE), methylation-sensitive restriction Endonuclease-PCR/Southern (MSRE-PCR/Southern), MethyLight, Pyrosequencing, combined bisulfite restriction analysis (COBRA), methylation-specific PCR (MSP), bisulfite sequencing, high resolution melting (HRM), Restriction Landmark Genomic Scanning (RLGS), amplification of inter-methylated sites (AIMS), Methylated CpG-island amplification (MCA), Differential Methylation Hybridization (DMH), HpaII tiny fragment Enrichment by Ligation-mediated PCR (HELP) and Methylated DNA immunoprecipitation (MeDIP).

4. The method of claim 1, wherein the biological sample is selected from the group consisting of blood, serum, plasma, sputum, saliva, kidney biopsy tissue and urine.

5. The method of claim 1, wherein the subject is of Asian descent, preferably a Chinese.

6. The method of claim 1, wherein if the total DNA methylation level is higher or lower than the corresponding total level in a standard control, the method further comprising administering to the subject agents for reducing blood glucose and urine protein, optionally, the standard control is a corresponding biological sample obtained from a healthy subject having no diabetes.

7. A method for determining a total methylation level of one or more CpG sites in a subject, the method comprising:
(a) extracting DNA from a biological sample obtained from the subject;
(b) performing an assay by contacting the DNA with reagents hybridizing to the one or more CpG sites, wherein the one or more CpG sites are selected from the group consisting of those given by CpG site number provided in Table 4;
(c) detecting a total number of the one or more CpG sites based on the signals obtained from the assay;
(d) determining the total methylation level of the one or more CpG sites using the total number.

8. The method of claim 7, wherein in step (b), the one or more CpG sites are selected from the group consisting of those having a positive value of the Model coefficient in Table 4, and if the total DNA methylation level is lower than the corresponding total level in a standard control, the method further comprising administering to the subject agents for reducing blood glucose and urine protein, optionally, the standard control is a corresponding biological sample obtained from a healthy subject having no diabetes.

9. The method of claim 7, wherein in step (b), the one or more CpG sites are selected from the group consisting of those having a negative value of the Model coefficient in Table 4, and if the total DNA methylation level is higher than the corresponding total level in a standard control, the method further comprising administering to the subject agents for reducing blood glucose and urine protein, optionally, the standard control is a corresponding biological sample obtained from a healthy subject having no diabetes.

10. The method of claim 7, wherein the subject has already had diabetes, such as type 1 diabetes (T1D) or type 2 diabetes (T2D).

11. The method of claim 7, wherein the reagents hybridizing to the one or more CpG sites are those involved in methods selected from the group consisting of High-performance Liquid Chromatography (HPLC), High-performance Capillary Electrophoresis (HPCE), methylation-sensitive restriction Endonuclease-PCR/Southern (MSRE-PCR/Southern), MethyLight, Pyrosequencing, combined bisulfite restriction analysis (COBRA), methylation-specific PCR (MSP), bisulfite sequencing, high resolution melting (HRM), Restriction Landmark Genomic Scanning (RLGS), amplification of inter-methylated sites (AIMS), Methylated CpG-island amplification (MCA), Differential Methylation Hybridization (DMH), HpaII tiny fragment Enrichment by Ligation-mediated PCR (HELP) and Methylated DNA immunoprecipitation (MeDIP).

12. The method of claim 7, wherein the biological sample is selected from the group consisting of blood, serum, plasma, sputum, saliva, kidney biopsy tissue and urine.

13. The method of claim 7, wherein the subject is of Asian descent, preferably a Chinese.

14. A method for calculating a baseline eGFR or an eGFR slope in a subject, comprising:
(a) extracting DNA from a biological sample obtained from the subject;
(b) performing an assay by contacting the DNA with reagents hybridizing to two or more CpG sites, wherein the two or more CpG sites are selected from the group consisting of those given by CpG site number provided in Tables 5-6;
(c) detecting a respective number of the two or more CpG sites based on the signals obtained from the assay;
(d) determining a respective methylation level of the two or more CpG sites using the respective number; and
(e) using the respective methylation level of each CpG site multiplying respective model coefficient of the CpG site and adding up together, and optionally plus the respective intercept shown in Supplementary Tables 5-6, to calculate the baseline eGFR or an eGFR slope.

15. The method of claim 14, wherein for the baseline eGFR, the two or more CpG sites are selected from the group consisting of those given by CpG site number provided in Tables 5 and the respective model coefficient is selected from the group consisting of that shown in “with covariates” and that shown in “without covariates” corresponding to each CpG sites shown in Table 5, and/or for the eGFR slope, two or more CpG sites are selected from the group consisting of those given by CpG site number provided in Table 6 and the respective model coefficient is selected from the group consisting of that shown in “with covariates” and that shown in “without covariates” corresponding to each CpG sites shown in Table 6.

16. The method of claim 15, wherein the method further comprises comparing the baseline eGFR or the eGFR slope to a cutoff, and wherein if the baseline eGFR or the eGFR slope is below the cutoff, the method further comprising administering to the subject agents for reducing blood glucose and urine protein.

17. The method of claim 15, wherein the subject has already had diabetes, such as type 1 diabetes (T1D) or type 2 diabetes (T2D).

18. The method of claim 15, wherein the reagents hybridizing to the one or more CpG sites are those involved in methods selected from the group consisting of High-performance Liquid Chromatography (HPLC), High-performance Capillary Electrophoresis (HPCE), methylation-sensitive restriction Endonuclease-PCR/Southern (MSRE-PCR/Southern), MethyLight, Pyrosequencing, combined bisulfite restriction analysis (COBRA), methylation-specific PCR (MSP), bisulfite sequencing, high resolution melting (HRM), Restriction Landmark Genomic Scanning (RLGS), amplification of inter-methylated sites (AIMS), Methylated CpG-island amplification (MCA), Differential Methylation Hybridization (DMH), HpaII tiny fragment Enrichment by Ligation-mediated PCR (HELP) and Methylated DNA immunoprecipitation (MeDIP).

19. The method of claim 15, wherein the biological sample is selected from the group consisting of blood, serum, plasma, sputum, saliva, kidney biopsy tissue and urine.

20. The method of claim 15, wherein the subject is of Asian descent, preferably a Chinese.
